(19) United States
(12) Patent Application Publication
(10) Pub. No.: US 2023/0151283 A1
(43) Pub. Date: May 18, 2023

(54) HYDROCARBON PYROLYSIS OF FEEDS CONTAINING NITROGEN

(71) Applicant: ExxonMobil Chemical Patents Inc., Baytown, TX (US)

(72) Inventors: Mark A. Nierode, Kingwood, TX (US); Rodney S. Smith, Edinburgh (GB); Donald J. Norris, London (CA); Asit K. Mondal, Yanbu (SA); Roshni Jindal, Singapore (SG)

(21) Appl. No.: 17/995,362

(22) PCT Filed: Mar. 12, 2021
(86) PCT No.: PCT/US2021/022231

§ 371 Date: Oct. 3, 2022

Related U.S. Application Data
(60) Provisional application No. 63/012,891, filed on Apr. 20, 2020.

(30) Foreign Application Priority Data
Jul. 6, 2020 (WO) 20184289.5

(51) Int. Cl. C10G 55/04; C10G 9/36; C07C 4/04; C07C 11/09; C07C 41/06; C10G 17/02; C10G 21/20; C10G 31/08; C10G 19/02

(52) U.S. Cl. CPC C10G 55/04; C10G 9/36; C07C 4/04; C07C 11/09; C07C 41/06; C10G 17/02; C10G 21/20; C10G 31/08; C10G 19/02; C10G 2300/201; C10G 2300/44; C10G 2400/20; C10G 2300/805

(57) ABSTRACT

The invention relates to hydrocarbon pyrolysis, e.g., the steam cracking of feeds comprising hydrocarbon and nitrogen-containing compositions. The invention also relates to equipment, systems, and apparatus useful for such pyrolysis, to the products and by-products of such pyrolysis, and to the further processing of such products and co-products, e.g., by polymerization.

### CROSS-REFERENCE OF RELATED APPLICATIONS

This application claims priority to and the benefit of U.S. Provisional Application No. 63/012,891, filed Apr. 20, 2020 and EP Application No. 20184289.5, filed Jul. 6, 2020, the disclosures of which are incorporated herein by reference in their entirety.

### FIELD

The invention relates to hydrocarbon pyrolysis, e.g., the steam cracking of feeds comprising hydrocarbon and nitrogen-containing compositions. The invention also relates to equipment, systems, methods, and apparatus useful for such pyrolysis, to the products and by-products of such pyrolysis, and to the further processing of such products and co-products, e.g., by polymerization.

### BACKGROUND

A variety of refinery process streams can be produced by processing raw feeds such as crude oil. Many of these refinery process streams are utilized as (and/or included in) feeds for hydrocarbon pyrolysis processes such as steam cracking. Steam cracking produces useful products such as light olefin from feeds (“steam cracker feeds”) comprising hydrocarbon (“hydrocarbon feeds”) and steam. Besides molecular hydrocarbon, saturated hydrocarbon, and water, steam cracking produces unsaturated products, e.g., olefins, such light (C₄₋) olefins including ethylene and propylene. Steam cracking also produces steam cracker tar, which can be used as a fuel oil), pyrolysis gasoline, steam cracker gas oil, etc.

Some of refinery streams that are used as the hydrocarbon feed for a steam cracking process are primarily vapor phase at a temperature of 25° C. and a pressure of 1 bar (abs). Others are primarily liquid phase under these conditions, e.g., refinery streams such as naphtha, gas oil, resids, etc. Besides those available from refining processes, primarily liquid-phase hydrocarbon feeds may be obtained from other petrochemical facilities, or from sources such as pipelines, transport vessels, tankage, etc. An advantage of obtaining such feeds from refining processes is that the refining processes used to produce the hydrocarbon feed typically remove various forms of nitrogen (e.g., N₂, and other nitrogen-containing compositions such as nitrogen compounds) that are typically present in refinery feed. For example, in many refinery product streams nitrogen is present as ammonia.

Over time, demand growth for light olefin has exceeded that of refinery products (e.g., fuels and lubricating oils), and this trend is expected to continue. As a result, both the number and size of new or revamped steam cracker plants have exhibited a significant increase in comparison with the number and size of new or revamped refineries. The resulting demand increase for primarily liquid-phase hydrocarbon feeds has increased interest in utilizing heavier liquid-phase feeds, e.g., those primarily liquid-phase hydrocarbon feedstocks having an API gravity less than that of naphtha (“relatively-heavy primarily liquid-phase hydrocarbon feeds”, also called “advantaged feeds”). Although advantaged feeds can include those that have been subjected to prior processing, such as certain gas oils, advantaged feeds also can include raw feeds such as crude oils, e.g., crude oils comprising medium hydrocarbon and/or heavy hydrocarbon. For example, utilizing advantaged feeds comprising raw feedstocks, e.g., various crude oils, would increase the supply of available liquid feeds, and would decrease the steam cracker plant's dependence on refinery process streams to satisfy steam cracker feed needs. This in turn would improve plant economics, e.g., by decreasing light olefin production costs, and by making relatively high-value refinery streams available for other purposes.

The amount of nitrogen (in various forms, e.g., as contaminants, contained in advantaged feeds can be an obstacle to utilizing them for steam cracking. Utilizing a raw feed comprising nitrogen-containing compositions, such as crude oil, can lead to processing difficulties, e.g., the degradation of a steam cracker system's catalysts and other components by acetonitrile and/or other nitrogen-containing contaminants. For example, nitrogen-containing compositions can poison catalysts, such as acetylene converter catalysts, methyl acetylene and propadiene (MAPD) converter catalysts, pyrolysis gasoline hydroprocessing catalysts, acidic catalysts such as those used for MTBE production, and other catalysts and/or catalytic beds. Steam cracker system components can also be corroded by ammonium salts and/or amine salts. A relatively high pH in steam cracker process streams resulting from ammonia and/or amine can lead to oil-in-water issues. This in turn can result in equipment fouling and a decrease in catalyst performance. The formation of NOx in various process stream associated with steam cracking can undesirably affect cryogenic equipment and furnace emissions.

The presence of nitrogen-containing compositions in steam cracker feed can be a particular difficulty in producing the desired light olefin products. Product specifications for the permissible amounts of ammonia, amine, nitriles, and other nitrogen-containing compositions compounds in ethylene and/or propylene streams can be very stringent, e.g., less than 1 parts per million by weight (“wppm”) of total nitrogen-containing compositions in ethylene and/or propylene grades utilized for producing polymeric products, such as polyethylene and polypropylene. Utilizing feeds containing an appreciable amount of various forms of nitrogen can lead to difficulties achieving light olefin streams of the specified purity.

Conventional methods have been proposed for removing nitrogen-containing compositions from hydrocarbon feeds before steam cracking is carried out. One such method, feed hydroprocessing, can remove certain nitrogen-containing compositions, but this method is costly and typically results in undesirable conversion of feed hydrocarbon compounds products of lesser value such as methane. Another conventional method utilizes a flash separation vessel integrated with a steam cracking furnace. This method removes and conducts away at least some of the hydrocarbon feed's nitrogen-containing compositions before steam cracking is carried out in the furnace's radiant section. Further improvements are needed, however, as limits on the amount of nitrogen-containing compositions in steam cracker product become increasingly stringent.

In particular, improved systems, methods, and processes are needed to manage nitrogen-containing compositions found in advantaged feeds or produced by the steam cracking of advantaged feeds, e.g. raw feeds such as crude oil. It is desired to efficiently manage nitrogen-containing compositions in hydrocarbon feeds for steam cracking in order to: (i) meet increasingly stringent product specifications; (ii) decrease operational costs of the steam cracking plant, e.g., those associated with catalyst poisoning in the plant's recovery facility; and/or (iii) reduce operating costs associated with corrosion from nitrogen-containing compositions such as ammonia.

### SUMMARY

Certain aspects of the invention are disclosed which provide, processes, methods, and apparatus for producing light olefin and lessening or eliminating undesirable effects resulting from the presence of various nitrogen0containing compositions in steam cracker feeds containing heavy hydrocarbons, as well as from other hydrocarbon streams and feeds.

The invention is based in part on the discovery that for a wide range of hydrocarbon feeds, particularly heavy feeds, the presence of various nitrogen-containing compositions in the feed results in the appearance of nitrogen-containing compositions in streams that are separated from the steam cracker effluent. It is observed that these separated streams can include nitrogen-containing compositions of the feed that are carried through the steam cracking process and into the steam cracker effluent and/or nitrogen-containing compositions that are derived from those of the feed, e.g., are converted from forms of nitrogen in the feed to the same forms or to other forms of nitrogen in the products of the steam cracking.

Accordingly, certain aspects of the invention relates to methods for steam cracking a hydrocarbon feed comprising hydrocarbon and a first nitrogen material. The hydrocarbon feed is cracked in a steam cracking furnace to produce a steam cracker effluent. A steam cracker tar and an upgraded steam cracker effluent are separated from the steam cracker effluent. The method also includes separating from the upgraded steam cracker effluent (i) a process gas comprising a second nitrogen material and (ii) a Pygas comprising a third nitrogen material, wherein the second and third nitrogen materials are each a portion of the first nitrogen material and/or are each derived from a portion of the first nitrogen material. A concentrated Pygas stream and a separated water stream containing at least a portion of the third nitrogen material are separated from the Pygas stream. A light effluent and a remaining water component are separated from the separated water stream, wherein the light effluent comprises at least a portion of the third nitrogen material. At least a portion of the third nitrogen material in the light effluent is removed to produce a purified light effluent.

In other aspects, the invention relates to methods for producing light olefins from a feed comprising heavy hydrocarbon and a first nitrogen material by steam cracking. The method includes separating from the steam cracker effluent a steam cracker tar and an upgraded steam cracker effluent, and separating from the upgraded steam cracker effluent at least (i) a process gas comprising a second nitrogen material and (ii) a Pygas comprising a third nitrogen material, wherein the second and third nitrogen materials are each a portion of the first nitrogen material and/or are each derived from a portion of the first nitrogen material. The process gas is transferred through a compressor and a condenser and into a knockout drum to produce a compressed process gas comprising first portion of the process gas's second nitrogen material, a hydrocarbon-water mixture, and a purge fluid comprising a second portion of the process gas's second nitrogen material. The compressed process gas is flowed through an amine tower and a caustic tower to produce a purified process gas. Various useful products and coproducts can be recovered from the purified process gas, e.g., polymer grade light olefin.

In certain aspects, a method includes introducing a hydrocarbon feed to a steam cracker to produce a steam cracker effluent, introducing the steam cracker effluent to a tar knock-out drum and separating a steam cracker tar from a upgraded steam cracker effluent, and introducing the upgraded steam cracker effluent to a fractionator and a quench tower to produce at least a Pygas stream and a process gas. The method also includes transferring the process gas through a compressor and a condenser and into a knockout drum to produce a treated light hydrocarbons stream, a hydrocarbon-water mixture, and a purge fluid containing the nitrogen contaminant, and flowing the treated light hydrocarbons stream through an amine tower and a caustic tower to produce a caustic treated stream containing a light hydrocarbon product.

In other aspects, the invention relates to systems and apparatus for steam cracking hydrocarbon feeds comprising nitrogen-containing compositions, e.g., systems and apparatus for carrying out any of the preceding methods and processes. In certain of these aspects, a steam cracker includes a convection line and a radiant line disposed within the convection line, a flash separation vessel fluidly coupled to and downstream of the convection line and fluidly coupled to and downstream of the radiant line, and a tar knock-out drum fluidly coupled to and downstream of the radiant line. A fractionator fluidly coupled to and downstream of the tar knock-out drum us also included, as are a quench tower fluidly coupled to and downstream of the fractionator, an oil and water separator fluidly coupled to and downstream of the quench tower, and a water stripper fluidly coupled to and downstream of the oil and water separator, where the water stripper has an overhead which is fluidly coupled to and upstream of a condenser and a vessel by a first line and fluidly coupled to and upstream of the quench tower by a second line.

### BRIEF DESCRIPTION OF THE DRAWINGS

So that the manner in which the above recited features of the disclosure can be understood in detail, a more particular description of the disclosure, briefly summarized above, may be had by reference to implementations, some of which are illustrated in the appended drawings. It is to be noted, however, that although the appended drawings illustrate typical implementations of this disclosure, these are not to be considered limiting of scope, for the disclosure may admit to other effective implementations.

FIG. 1 depicts a partial schematic view of a process system for producing light olefins that includes a hydrocarbon steam cracking and fractioning system, according to one or more aspects.

FIG. 2 depicts another partial schematic view of the process system illustrated in FIG. 1, which includes a Pygas and water separation and purification system, according to one or more aspects.

FIG. 3A depicts another partial schematic view of the process system illustrated in FIG. 1, which includes a light hydrocarbon recovery system, according to one or more aspects.

FIG. 3B depicts a partial schematic view of the light hydrocarbon recovery system illustrated in FIG. 3A, according to one or more aspects.

To facilitate understanding, identical reference numerals have been used, where possible, to designate identical elements that are common to the Drawings. It is contemplated that elements and features of one implementation may be beneficially incorporated in other implementations without further recitation.

### DETAILED DESCRIPTION

In certain aspects, the invention relates to methods and apparatus for removing nitrogen-containing compositions such as ammonia from various locations in steam cracker processes. The nitrogen in such compositions can be present in various forms, e.g., as one or more nitrogen-containing compounds, etc. The management of nitrogen-containing compositions at various locations in a steam cracking process improves process efficiency and cost-effectiveness, and provides products and co-products that meet increasingly stringent specifications.

Certain aspects of the invention are carried out in a steam cracker plant comprising a furnace facility and a recovery facility. The furnace facility typically includes at least one at least one steam cracking furnace that is configured for pyrolysing the feed. The steam cracking furnace typically includes a convection section, a radiant section, and a vapor-liquid separator that is generally integrated with the convection section. Various products and co-products are recovered from the steam cracker effluent in a recovery facility located downstream of the steam cracking facility. The recovery facility can include one or more vessels (e.g., a flash drum, such as a tar-knock-out drum), for separating from the steam cracker effluent a steam cracker tar and an upgraded steam cracker effluent. A primary fractionator is typically used for separating quench oil, gas oil, etc. from the upgraded steam cracker effluent. A vapor stream conducted away from the primary fractionator overhead is typically quenched in at least one vessel (e.g., a quench tower) for recovery of a naphtha boiling-range composition (e.g., pyrolysis gasoline), water, and a process gas. Optionally, the primary fractionator can be combined with the quench tower. Additional product separation and recovery equipment is typically used, e.g., for recovering ethylene and/or propylene. The invention is not limited to these aspects, and this description should not be interpreted as foreclosing other aspects of pyrolysis and product/co-product recovery within the broader scope of the invention

### Definitions

“Hydrocarbon” means a class of compounds containing hydrogen bound to carbon. The term “Cₙ” hydrocarbon means hydrocarbon having n carbon atom(s) per molecule, where n is a positive integer. The term “Cₙ₊” hydrocarbon means hydrocarbon having at least n carbon atom(s) per molecule, where n is a positive integer. The term “Cₙ₋” hydrocarbon means hydrocarbon having no more than n number of carbon atom(s) per molecule, where n is a positive integer. “Hydrocarbon” encompasses (i) saturated hydrocarbon, (ii) unsaturated hydrocarbon, and (iii) mixtures of hydrocarbons, including mixtures of hydrocarbon compounds (saturated and/or unsaturated), including mixtures of hydrocarbon compounds having different values of n. The term “unsaturate” or “unsaturated hydrocarbon” means a C₂₊ hydrocarbon containing at least one carbon atom directly bound to another carbon atom by a double or triple bond. The term “olefin” means an unsaturated hydrocarbon containing at least one carbon atom directly bound to another carbon atom by a double bond. In other words, an olefin is a compound which contains at least one pair of carbon atoms, where the first and second carbon atoms of the pair are directly linked by a double bond. “Light olefin” means C₅₋ olefinic hydrocarbon.

“Heavy hydrocarbon” means a mixture comprising hydrocarbon, the mixture having an API gravity in the range of from 5° up to (but not including) 22°. “Medium hydrocarbon” means a mixture comprising hydrocarbon, the mixture having an API gravity in the range of from 22° to 30°. A “relatively-heavy” hydrocarbon has an API gravity that is less than that of naphtha. A “sour” hydrocarbon is a hydrocarbon e.g., a crude oil, comprising ≥0.5 wt. % of sulfur based on the weight of the hydrocarbon, where the weight percent encompasses all forms of sulfur in the hydrocarbon, e.g., one or more of elemental sulfur, sulfur bound in compounds, sulfur bound to, entangled with, or associated with aggregates such as asphaltenes and tar heavies, etc.

Certain medium and/or heavy hydrocarbons, e.g., certain raw hydrocarbon feedstocks, such as certain crude oils and crude oil mixtures, contain one or more of asphaltenes, precursors of asphaltenes, and particulates. Asphaltenes are described in U.S. Pat. No. 5,871,634, which is incorporated herein by reference in its entirety. Asphaltene content can be determined using ASTM D6560-17. “Resid” means an oleaginous mixture, typically contained in or derived from crude oil, the mixture having a normal boiling point range ≥1050° F. (566° C.). Resid can include non-volatile components, meaning compositions (organic and/or inorganic) having a normal boiling point range ≥590° C. Certain non-volatile components have a normal boiling ≥760° C. “Raw” feedstock, e.g., raw hydrocarbon feedstock, means a primarily liquid-phase feedstock that comprises ≥25 wt. % of crude oil that has not been subjected to prior desalting and/or to prior fractionation with reflux, e.g., ≥50 wt. %, such as ≥75 wt. %, or ≥90 wt. %. “Crude oil” means a mixture comprising naturally-occurring hydrocarbon of geological origin, where the mixture (i) comprises ≥1 wt. % of resid, e.g., ≥5 wt. %, such as ≥10 wt. %, and (ii) has an API gravity ≤52°, e.g., ≤30°, such as ≤20°, or ≤10°, or <8°. The crude oil can be classified by API gravity, e.g., heavy crude oil has an API gravity in the range of from 5° up to (but not including) 22°. Likewise, a medium crude oil has an API gravity in the range of from 22° to 30°.

“Primarily liquid phase” means a composition of which ≥50 wt. % is in the liquid phase, e.g., ≥75 wt. %, such as ≥90 wt. %. A hydrocarbon feed is primarily liquid-phase when ≥50 wt. % of the hydrocarbon feedstock is in the liquid phase at a temperature of 25° C. and a pressure of 1 bar absolute, e.g., ≥75 wt. %, such as ≥90 wt. %.

Normal (or “atmospheric”) boiling points and normal boiling point ranges can be measured by gas chromatograph distillation according to the methods described in ASTM D-6352-98 or D2887, as extended by extrapolation for materials above 700° C. The term “T₅₀” means a temperature, determined according to a boiling point distribution, at which 50 weight percent of a particular sample has reached its boiling point. Likewise, “T₉₀”, “T₉₅” and “T₉₈” mean the temperature at which 90, 95, or 98 weight percent of a particular sample has reached its boiling point. Nominal final boiling point means the temperature at which 99.5 weight percent of a particular sample has reached its boiling point.

A “steam cracker” is a form of thermal pyrolysis apparatus having at least a convection section and a radiant section. The term “steam cracker” is interchangeable with “thermal pyrolysis unit”, “pyrolysis furnace”, “steam cracking furnace”, or just “furnace.” Steam, although optional, may be added for a variety of reasons, such as to reduce hydrocarbon partial pressure, to control residence time, and/or to decrease coke formation. In certain aspects, the steam may be superheated, such as in the convection section of the furnace, and/or the steam may be sour or treated process steam. Heat for the furnace is provided by burners located in the radiant section. The burners combust fuel and air, and produce a flow of combustion effluent. The combustion effluent flows out of the radiant section, through the convection section, and is then conducted away from the steam cracking furnace. The convection section includes at least one tubular member (a “convection coil”). Likewise, the radiant section also includes at least one tubular member (a “radiant coil”). The outer surface of the radiant coil is heated at least by radiant heat from the burners. The outer surface of the convection coil is heated at least by combustion effluent traversing the convection section. The downstream end of the convection coil is in fluidic communication with the upstream end of the radiant coil via crossover piping. At least one vapor-liquid separator can be integrated with the convection section, e.g., in fluidic communication with the convection coil and/or crossover piping. A feed comprising hydrocarbon and various nitrogen-containing compositions (a “hydrocarbon feed”) is introduced into the convection coil for preheating, typically after desalting. Steam is added to the preheated feed to produce a steam cracking feed. The steam may be superheated, such as in the convection section of the furnace, and/or the steam may be sour or treated process steam. A primarily vapor-phase pyrolysis feed and a primarily liquid bottoms stream can be separated from the preheated feed, e.g., in the vapor-liquid separator. The pyrolysis feed is conducted into the radiant coil, typically via crossover piping, and optionally after heating in one or more additional convection coils. A steam cracker effluent is conducted away from the radiant coil outlet. To lessen the amount of over-cracking and other undesired side-reactions, the steam cracker effluent is rapidly cooled (“quenched”), e.g., by indirect cooling in one or more heat exchangers (such as one or more transfer line exchangers) and/or direct cooling by injecting of a quench fluid, e.g., one or more of an oleaginous quench fluid such as quench oil, liquid water, and steam. The addition of steam at various points in the process is not detailed in every embodiment described. It is further noted that any of the steam added may include untreated or treated process steam and that any of the steam added, whether treated or not, may be superheated. For example, superheating the stream can be performed when the steam is produced from sour water.

“Pygas” means pyrolysis gasoline (also called steam cracker naphtha, “SCN”), which is a mixture derived (e.g., by one or more separations) from a pyrolysis effluent (such as a steam cracker effluent) and composing hydrocarbons having normal boiling points in what's conventionally referred to as the “naphtha boiling range”, e.g., an atmospheric boiling point range of from an initial boiling point of about 30° F. (1.1° C.) to about 500° F. (260° C.), such as from about 40° F. (4.4° C.) to about 450° F. (232° C.), or from about that of mixed C₅ hydrocarbon to 430° F. (221° C.). Pygas typically comprises C₅₊ hydrocarbons, e.g., C₅-C₁₀₊ hydrocarbons, having an initial atmospheric boiling point of about 25° C. to about 50° C. and a final boiling point of about 220° C. to about 265° C., as measured according to ASTM D2887-18. In some examples, Pygas has an initial atmospheric boiling point of about 33° C. to about 43° C. and a final atmospheric boiling point of about 234° C. to about 244° C., as measured by ASTM D2887-18.

“Steam cracker tar” (or “SCT”) means a mixture comprising (i) aromatics and optionally (ii) non-aromatics and/or non-hydrocarbons, the mixture being derived from hydrocarbon pyrolysis and having a T₉₀≥290° C., e.g., ≥500° C., such as ≥600° C., or greater. In certain aspects, SCT is separated from quench (or partially quenched) steam cracker effluent in a separation vessel such as tar knock-out drum, primary fractionator, etc. SCT can include hydrocarbon molecules (including mixtures and aggregates thereof) having (i) one or more aromatic components and (ii) a molecular weight of about C₁₅ or greater of about 50 wt. % or greater (e.g., 75 wt. % or greater, such as 90 wt. % or greater), based on the weight of the SCT.

The forms of nitrogen-containing compositions present in hydrocarbon feed and in products, co-products, by-products, and other streams and compositions associated with steam cracking can be or include, e.g., one or more of ammonia, ammonium or one or more ammonium cations or compounds, one or more amine, one or more nitriles, hydrogen cyanide, one or more NOₓ compounds, NOₓ compounds ions, and NOₓ compounds salts. The term “amine” means compounds and functional groups that contain a basic nitrogen atom with a lone pair (i.e., unshared pair or non-bonding pair) of valence electrons, and encompasses all primary, secondary, and tertiary amines Ammonium cations or compounds can be or include, e.g., one or more of those cations or compounds having the chemical formula [RₓNH₍₄₋ₓ₎]⁺, where x is 0, 1, 2, 3, or 4, and each R is independently selected from among alkyl, aryl (such as phenyl), or other organic groups. Exemplary ammonium cations or compounds can be or include, e.g., one or more of ammonium, methylammonium, tetramethylammonium, ethylammonium, and salts of any of these. The term “amine” means compounds and functional groups that contain a basic nitrogen atom with a lone pair (i.e., unshared pair or non-bonding pair) of valence electrons, and encompasses all primary, secondary, and tertiary amines Amine can be or include, e.g., one or more of those having the chemical formula RₓNH₍₃₋ₓ₎, where x is 1, 2, or 3, and each R is independently selected from among alkyl, aryl (such as phenyl), or other organic groups. Exemplary amine can be or include, e.g., one or more of methylamine, dimethylamine, trimethylamine, ethylamine, diethylamine, triethylamine, methylethylamine (MEA), phenylamine, and salts of any of these. Nitriles can be or include one or more of those having the chemical formula RCN, where R is an alkyl, an aryl (such as phenyl), or other organic group. Exemplary nitriles can be or include one or more of acetonitrile, ethanenitrile, propanenitrile, benzonitrile, and derivatives of any of these. Exemplary nitrogen oxide (NOₓ) compounds (or ions of such compounds) can be or include, e.g., one or more of nitric oxide (NO), dinitrogen oxide (N₂O), dinitrogen dioxide (N₂O₂), nitrogen dioxide (NO₂), nitrogen pentoxide (NO₅), dinitrogen pentoxide (N₂O₅), and ions of any of these.

Unless the context expressly indicates otherwise, the amount of a particular nitrogen-containing composition in a particular stream, e.g., hydrocarbon feed, products, co-products, by-products, and other streams and compositions associated with steam cracking, is the total mass of all nitrogen atoms (including, e.g., the mass of nitrogen atoms in aggregates, mixtures, compounds, complexes, etc.) relative to the total mass of the stream. This is typically expressed as a weight percent of the stream. The amount of nitrogen within each nitrogen-containing composition is the weight of nitrogen atoms based on the weight of the composition, and is typically expressed as a weight percent of the composition. Any suitable technique can be used to determine the amount of nitrogen atoms in a particular composition, including conventional techniques or by reference to published tabulations and compendiums. The term “nitrogen material” means all the various forms of nitrogen-containing compositions, e.g., one or more of aggregates, mixtures, compounds, complexes, etc. The term nitrogen material encompasses natural and synthetic forms of nitrogen. Those skilled in the art will appreciate that depending on the context the nitrogen material of a particular stream can mean one form of nitrogen, e.g., ammonia, or a plurality of nitrogen forms. Unless otherwise expressly indicated in a particular context, the amount of nitrogen material in a process stream, e.g., the amount of nitrogen material present in its various forms in a stream, means the total mass of all forms of nitrogen present in a given mass of that stream, and is typically expressed as a weight percent based on the weight of that stream. Any suitable method can be used to determine the amount of nitrogen material in a particular stream, including conventional methods. When a second stream is said to have V % less nitrogen material than that of a first stream, where (i) the first stream comprises U wt. % of nitrogen material and (ii) U and V are real numbers ≥0, that means the amount of nitrogen material in the second stream is U wt. % minus (V % times U wt. %).

The term “non-volatile components” or “non-volatiles” means that portion of a composition, e.g., a hydrocarbon composition, having a nominal boiling point about 590° C. or greater, as measured by ASTM D-6352-98 or D-2887. Non-volatile components may be further limited to components with a boiling point of about 760° C. or greater. The boiling point distribution of a hydrocarbon stream may be measured by gas chromatograph distillation according to the methods described in ASTM D-6352-98 or D2887, extended by extrapolation for materials above 700° C. Non-volatile components may include coke precursors, which are moderately heavy and/or reactive molecules, such as multi-ring aromatic compounds, which can condense from the vapor phase and then from coke under the operating conditions encountered in one or more aspects of the invention.

In certain aspects the hydrocarbon feed comprises (i) nitrogen material and (ii) a heavy and/or medium hydrocarbon. These aspects will now be described in more detail. The invention is not limited to these aspects, and this description is not meant to exclude other aspects within the broader scope of the invention, such as those in which the hydrocarbon feed is a medium hydrocarbon.

### Hydrocarbon Feed

The hydrocarbon feed may include relatively high molecular weight hydrocarbons (heavy hydrocarbon), such as those which produce a relatively large amount of steam cracker naphtha (SCN), steam cracker gas oil (“SCGO”), and SCT during steam cracking. The heavy hydrocarbon typically includes C₅₊ hydrocarbon, which may include one or more of SCGO and residues, gas oils, heating oil, jet fuel, fuel oil, diesel, kerosene, coker naphtha, SCN, catalytically cracked naphtha, hydrocrackate, reformate, raffinate reformate, Fischer-Tropsch liquids, Fischer-Tropsch gases, distillate, crude oil, atmospheric pipestill bottoms, vacuum pipestill streams including bottoms, gas oil condensates, heavy non-virgin hydrocarbon streams from refineries, vacuum gas oils, heavy gas oil, naphtha contaminated with crude, atmospheric residue, heavy residue, C₄/residue admixture, naphtha residue admixture, gas oil residue admixture, low-sulfur waxy residue, atmospheric residue, and heavy residue. It may be advantageous to use a heavy hydrocarbon feedstock such as crude oil. Such heavy hydrocarbon feeds can include, e.g., economically advantaged, minimally processed heavy hydrocarbon streams containing non-volatile components and coke precursors. The hydrocarbon feed can have a nominal final boiling point of about 315° C. or greater, such as about 400° C. or greater, about 450° C. or greater, or about 500° C. or greater.

The hydrocarbon feed may include one or more relatively low molecular weight hydrocarbon (light hydrocarbon). Light hydrocarbon typically includes substantially saturated hydrocarbon molecules having fewer than five carbon atoms, e.g., ethane, propane, and mixtures thereof. Although hydrocarbon feeds of light hydrocarbon typically produce a greater yield of C₂ unsaturates (ethylene and acetylene) than do hydrocarbon feeds comprising heavy hydrocarbon, and the steam cracking of light hydrocarbon generally produces less SCN, SCGO, and SCT, the use of heavy hydrocarbon is of increasing interest due to lesser costs and greater availability. The relative amounts of light hydrocarbon (typically in the vapor phase) and heavy hydrocarbon (typically in the liquid phase) in the hydrocarbon feed can be from 100 wt. % light hydrocarbon to 100 wt. % heavy hydrocarbon, although typically there is about 1 wt. % or more heavy hydrocarbon present in a hydrocarbon feed. For example, the hydrocarbon feed can include about 1 wt. % or more of heavy hydrocarbon, based on the weight of the hydrocarbon feed, such as about 25 wt. % or more, about 50 wt. % or more, about 75 wt. % or more, about 90 wt. % or more, or about 99 wt. % or more.

Besides hydrocarbon, the hydrocarbon feed comprises nitrogen material. The nitrogen material can include one or more nitrogen-containing compositions. The amount of nitrogen as nitrogen atoms (including nitrogen in all forms that contain it) in the hydrocarbon feed is typically in the range of from about 20 parts per million by weight (wppm), about 50 wppm, about 100 wppm, about 150 wppm, or about 200 wppm to about 300 wppm, about 500 wppm, about 600 wppm, about 800 wppm, about 1,000 wppm, about 1,150 wppm, about 1,300 wppm, about 1,500 wppm, or greater.

### Desalter

In order to have a desirably low concentration of sodium in the radiant section of steam crackers, one or more desalters may be included to remove salts and particulate matter from the hydrocarbon feed prior to steam cracking. While acceptable amounts of salt and/or particulate matter can vary with furnace design and operating conditions, the addition of at least one desalter may be desired when ammonium salts, amine salts, alkali and/or rare earth salts (e.g., sodium chloride or magnesium carbonate), and/or other salts are present in the hydrocarbon feed in an amount greater than a few ppmw on a nitrogen atom basis for all salts that contain nitrogen Desalting removes salts and/or particulates to reduce catalyst poisoning, corrosion, fouling, and/or contamination issues. For example, by removing ammonium salts, amine salts, and/or other salts, corrosion issues of various downstream components and equipment throughout the process system can be decreased or eliminated.

In a typical desalting process, wash water (or fresh water, or deionized water) is mixed with a heated hydrocarbon feed to produce a water-in-oil emulsion, which in turn extracts salt, brine and particulates from the oil. The wash water used to treat the hydrocarbon feed may be derived from various sources and the water itself may be, for example, recycled refinery water, recirculated wastewater, clarified water, purified wastewater, sour water stripper bottoms, overhead condensate, boiler feed water, clarified river water or from other water sources or combinations of water sources. The amount of salts in water is expressed in parts per thousand of the salt by weight (ppt) and is based on the weight of the water. Typically the wash water can include from fresh water (less than 0.5 ppt), brackish water (0.5-30 ppt), saline water (greater than 30 ppt to 50 ppt) to brine (greater than 50 ppt). Although deionized water may be used to favor exchange of salt from the crude into the aqueous solution, deionized water is not normally required to desalt crude oil feedstocks although it may be mixed with recirculated water from the desalter to achieve a specific ionic or salt content in either the water before emulsification or to achieve a specific ionic strength in the desalter emulsion. Wash water rates may be from about 5% and about 7% by volume of the total crude charge, but may be higher or lower dependent upon the crude oil source and quality. A variety of water sources may be combined as determined by cost requirements, supply, salt content of the water, salt content of the hydrocarbon feed, and other factors specific to the desalting conditions such as the size of the separator and the degree of desalting required.

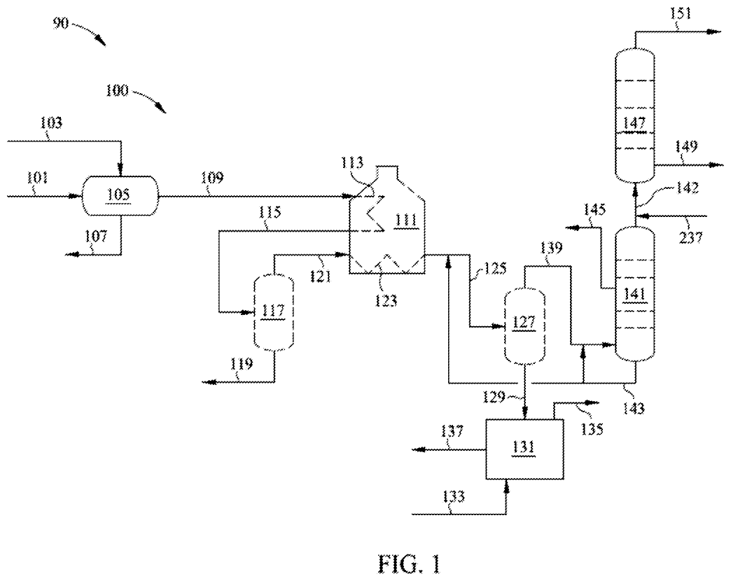

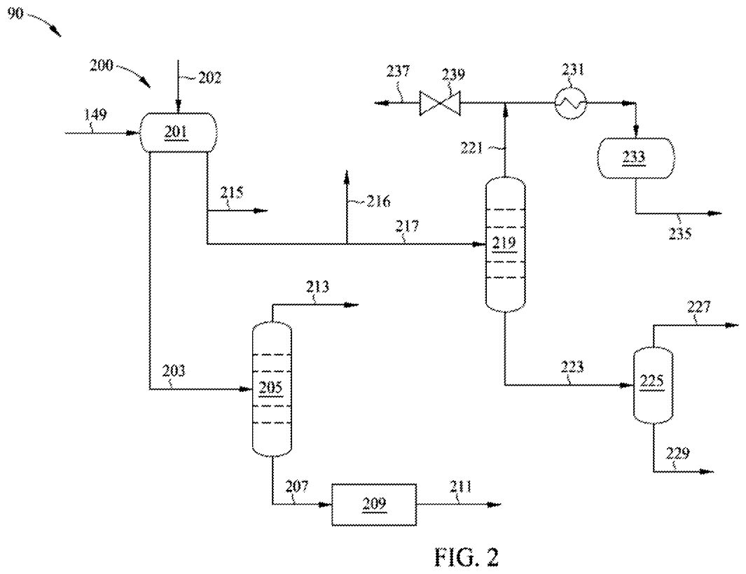

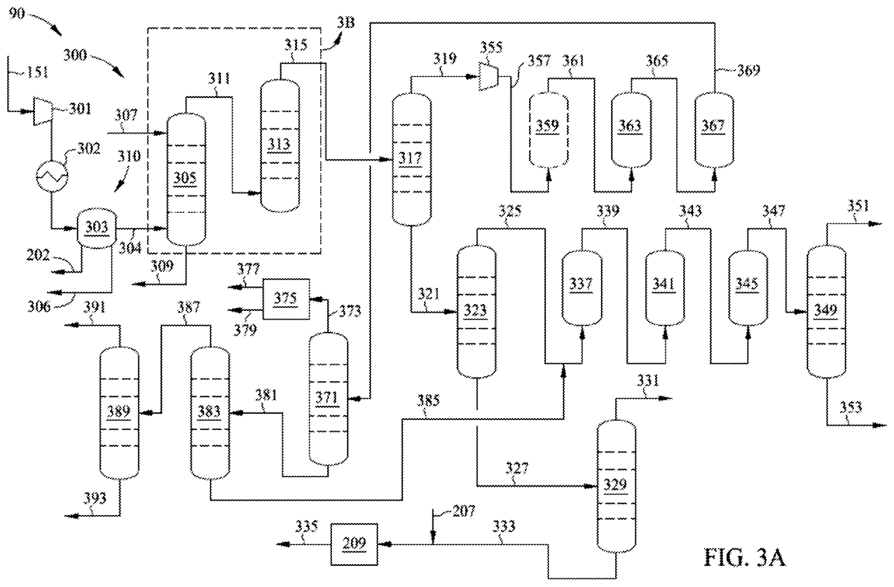

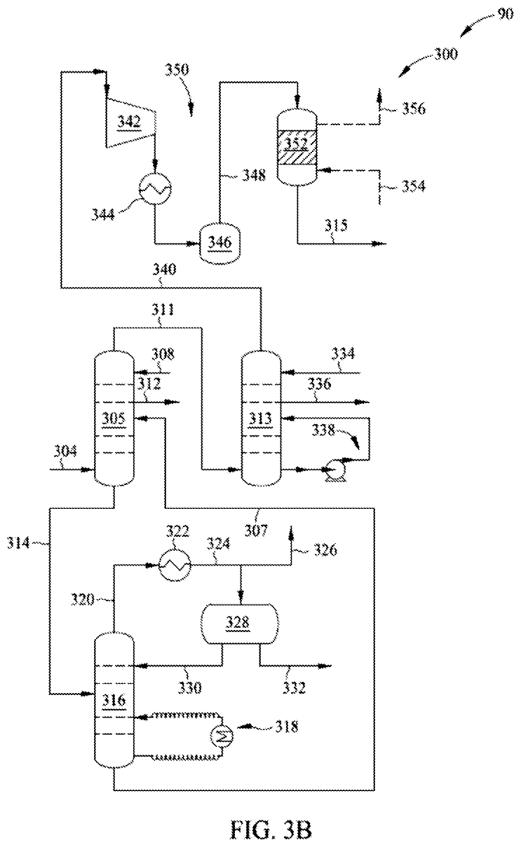

FIG. 1 depicts a partial schematic view of a pyrolysis process system 90 which is used to produce light olefins from a hydrocarbon feed 101. The pyrolysis process system 90 contains a steam cracking furnace and recovery systems 100, as depicted in FIG. 1, a Pygas and water separation and purification system 200, depicted in FIG. 2, and a light hydrocarbon recovery system 300, depicted in FIGS. 3A and 3B.

As depicted in FIG. 1, a salty emulsion is produced by combining a hydrocarbon feed 101 and wash water via line 103 within a desalter 105. Salt is separated from the hydrocarbon feed, producing (1) salt-enriched water that is transferred via line 107 and (2) desalted hydrocarbon feed that is removed from the desalter 105 via line 109. During the separation phase of the desalting process, an emulsion phase of varying composition and thickness may form at the interface of the oil and aqueous layers. An unrestricted growth in emulsion thickness can result in carry-over with the desalted crude oil (leading to equipment fouling) or carry-under into the aqueous layer (interferes with processing the salt-enriched water). Suitable countermeasures include, e.g., one or more of controlling emulsion formation and growth, removing the emulsions from desalter, and, using an additional processing step, and resolving the emulsion into its constituent parts (e.g., oil, water and solids) to allow for reuse and/or disposal of the oil, water, and solids.

Methods for resolving emulsions may include gravitational or centrifugal methods. In a gravity method, the emulsion is allowed to stand in the separator and the density difference between the oil and the water causes the water to settle through and out of the oil by gravity. In the centrifugation method, the stable emulsion is moved from the de-salter unit to a centrifuge (not shown) which separates the emulsion into separate water, oil and solids. The gravity method involves the use of time-intensive, and thus inefficient, settling tanks as well as costly methods for disposing of the partially resolved emulsion, while the centrifugation method involves large centrifuges that are costly to build and operate. Another method for resolving emulsions is the application of an electric field within the desalter. The application of an electric field may force water droplets to coalesce. Large electrocoalesced water droplets settle under gravity and penetrate through the oil/bulk-resolved-water interface to immerse into the resolved bulk water phase at the bottom of the desalter.

Certain hydrocarbon feed contain contaminants, including contaminants that are and/or contains one or more nitrogen-containing compositions. Some hydrocarbon feed contaminants, e.g., asphaltenes, resins, and finely divided solid particles (e.g., those having an average size of less than 5 microns). Typically at least some of these contaminants (e.g., ≥1% of these contaminants, such as ≥10%) can contain or are otherwise associated with one or more nitrogen-containing compositions. Certain of these contaminants, including those that contain or are otherwise associated with one or more nitrogen-containing compositions, can act as natural surfactants that stabilize the emulsion phase and cause the emulsion to persist in the desalter unit (the emulsion layer in the desalter is commonly referred to as the rag layer). The persistent emulsion problem is prevalent in the processing of a hydrocarbon feed including crude oil because of high solids content. Hydrocarbon feeds with high solids contents are typically not preferred since the presence of the solids, often with particle sizes under 5 microns, may act to stabilize the emulsion and the oil/bulk-resolved-water interface, leading to a progressive increase in the depth of the rag layer. The persistent existence of a rag layer may be due to the inability of electrocoalesced droplets to break the oil/bulk-resolved-water interface. The rag layer in the desalter typically contains a high concentration of oil, residual water, suspended solids and salts (including those containing nitrogen) which, in a typical example, might be about 70% v/v water, 30% v/v oil, with 5,000-8,000 pounds per thousand barrels (PTB) (about 14 g/L to about 23 g/L) solids, and 200-400 PTB (about 570 mg/L to about 1,100 mg/L) salts. The aqueous phase contains salts from the hydrocarbon feed, including salts that contain nitrogen.

One method of decreasing the size and effect of the persistent emulsified layer (rag layer) is the addition of demulsifiers. One suitable method for the addition of demulsifiers in the desalting process is described in U.S. Pub. No. 2016/0208176, incorporated by reference. Demulsifiers commonly used in the processing of hydrocarbon feeds that include heavy hydrocarbons may be useful in the desalting process although the desalting process may not be reliant on the specific demulsifier chosen. Demulsifiers may be one or more of: polyethyleneimines, polyamines, succinated polyamines, polyols, ethoxylated alcohol sulfates, long chain alcohol ethoxylates, long chain alkyl sulfate salts, e.g., sodium salts of lauryl sulfates, epoxies, di-epoxides (which may be ethoxylated and/or propoxylated). The addition of demulsifiers may be useful in the desalting of hydrocarbon feeds containing high levels of particulates or asphaltenes, which tend to stabilize the rag layer.

The desalted oil phase forms a top layer, which is continuously removed as desalted hydrocarbon feed via line 109 and the resolved bulk water accumulates in the bottom of the desalter and is continuously removed as salt-enriched water via line 107 (FIG. 1). The salt-enriched water may be sent for deionization and recycling or used with or without further processing in other processes.

### Steam Cracker

Steam cracking is carried out in at least one steam cracking furnace. The radiant section can include fired heaters (e.g., burners), and flue gas from combustion carried out with the fired heaters travels upward from the radiant section through the convection section and then away as flue gas. As shown in FIG. 1, desalted hydrocarbon feed via line 109 first enters a steam cracking furnace in the convection section (upper portion) and is sent through convection line 113 where it is preheated by indirect exposure to the flue gases in the convection section to produce a preheated feed. Preheated feed is mixed with dilution steam (not shown) to produce a steam cracking feed. The steam cracking feed is conducted via line 115 to flash separation vessel 117 (also referred to as a separation pot or vapor-liquid separator). A bottoms stream and a primarily vapor-phase pyrolysis feed are separated from the steam cracking feed in the separation vessel. The separated bottoms stream is conducted away vial line 119. The pyrolysis feed is transferred via line 121 to steam cracking furnace 111 and through one or more radiant lines 123 in the radiant section (lower portion) of the steam cracking furnace 111 for pyrolysis (cracking) to produce steam cracker effluent that is transferred to line 125 for further processing.

### Steam Cracker Convection Section

The desalted hydrocarbon feed (via line 109) is first preheated in the convection line 113 within the convection section of the steam cracking furnace 111. The preheating of the desalted hydrocarbon feed may include indirect contact (within the convection line) of the feed in the convection section of the steam cracker with hot flue gases from the radiant section of the furnace, e.g., by passing the desalted hydrocarbon feed through a bank of heat exchange tubes (also called convection coils) located within the convection section of the steam cracker. The preheated hydrocarbon feed may have a temperature from about 150° C. to about 260 C, such as about 160° C. to about 230 C, or about 170° C. to about 220° C.

The preheated hydrocarbon feed may be combined with steam (e.g., with dilution steam) and subjected to additional preheating in the convection coils. At least one diluent comprising steam is added to the desalted hydrocarbon feed to produce a steam cracking feed having steam amount in a range of from about 10 wt. % to about 90 wt. %, based on the weight of the steam cracking feed, with the ≥90 wt. % of the remainder of the steam cracking feed comprising the preheated hydrocarbon feed. In certain aspects, the weight ratio of steam to hydrocarbon in the steam cracking feed can be from about 0.1 to about 1, such as about 0.2 to about 0.6.

### Flash Separation Vessel

The stream cracker 111 may have integrated therewith one or more flash separation vessels 117, which is a vapor/liquid separation device (sometimes referred to as flash pot or flash drum), which can provide upgrading the preheated feed. Such flash separation vessels are suitable when the preheated feed includes about 0.1 wt. % or more of asphaltenes based on the weight of the hydrocarbon components of the convection product stream, e.g., about 5 wt. % or more. Upgrading the preheated feed through vapor/liquid separation may be accomplished through flash separation vessels or other suitable means. Examples of suitable flash separation vessels include those disclosed in U.S. Pat. Nos. 6,632,351; 7,090,765; 7,097,758; 7,138,047; 7,220,887; 7,235,705; 7,244,871; 7,247,765; 7,297,833; 7,311,746; 7,312,371; 7,351,872; 7,488,459; and 7,578,929; and 7,820,035, which are incorporated by reference herein.

Where a flash separation vessel is integrated with the steam cracker, at least a portion of the steam cracking feed is in the vapor phase. The steam cracking feed (via line 115) is transferred to and flashed in one or more flash separation vessels 117, in order to separate from the steam cracking feed (i) bottoms stream comprising at least a portion of the high molecular-weight molecules, such as asphaltenes, and (ii) a primarily vapor-phase pyrolysis feed. The bottoms stream can be conducted away from the flash separation vessel 117 as a by-product via line 119. The separated bottoms stream may include, for example, greater than about 10 wt. % of the asphaltenes in the preheated feed. The pyrolysis feed is conducted to the steam cracker 111 via line 121. Optionally, the pyrolysis feed is subjected to further indirect heating in additional convection coils (not shown), with the heated pyrolysis feed being conducted to radiant coils 123 via crossover piping (not shown).

Utilizing separation vessel 117 upstream of the radiant section can increase the breadth of hydrocarbon feeds available to be used directly, without pretreatment such as hydroprocessing, fractionation (e.g., fractionation with reflux), etc. Such a flash separation vessel can facilitate the processing of a hydrocarbon feed 101 that contains about 50 wt. % or greater heavy hydrocarbon (e.g., raw heavy hydrocarbon, such as crude oil), such as about 75 wt. % or greater, or about 90 wt. % or greater. Moreover, regulating the cut point of the flash separation vessel facilitates maintaining within desired limits the amounts of certain contaminants in the pyrolysis feed, e.g., the amounts of those comprising one or more nitrogen-containing compositions. Depending, e.g., on the selected separation conditions such as cut point temperature, pressure, flow rate of liquid in convection section coils located upstream of the separation vessel, etc., at least a portion (such as most or all) of any non-vapor-phase components in the steam cracking feed can be separated and conducted away with bottoms stream 119. Such non-vapor-phase components typically include salts and/or particulate matter, e.g., nitrogen-containing salts and/or nitrogen-containing particulate matter. Such non-vapor-phase components can also include at least a portion of any non-volatiles present in the steam cracking feed, e.g., ≥10 wt. % of any non-volatiles present in the steam cracking feed (based on the total weight of non-volatiles present in the steam cracking feed), such as ≥25 wt. %, or ≥50 wt. %, or ≥75 wt. %, or ≥90 wt. %. These features are particularly advantageous when <98 wt. % of the steam cracking feed's hydrocarbon is in the vapor phase at the inlet of flash separation vessel 117.

In certain aspects, a sufficient liquid velocity in those convection coils located upstream of the flash separation vessel 117 is maintained to keep at least a portion of non-vapor-phase components of the steam cracking feed (including any of these that comprise one or more nitrogen-containing compositions) in suspension until removed with bottoms stream 119. Doing so is observed to facilitate the separation from the stream cracking feed in the flash separation vessel 117 of non-vapor-phase components, e.g., salts and/or particulates, such as nitrogen-containing salts and/or nitrogen-containing particulates. Typically, the amount of the steam cracking feed that is separated and conducted away as bottoms stream 119 will vary with the properties and composition of the steam cracking feed's hydrocarbon component, the liquid velocity in those convection coils located upstream of the flash separation vessel, and the amount of non-vapor-phase components in the steam cracking feed. Maintaining the liquid velocity in the desired range can be achieved by regulating the amount of liquid-phase material in the steam cracking feed. A lesser amount of liquid-phase material in the steam cracker feed is needed to maintain the desired liquid velocity when the hydrocarbon component of the steam cracking feed includes viscous, generally heavier, liquid-phase hydrocarbon. Likewise, a greater amount of liquid-phase material in the steam cracker feed is needed to maintain the desired liquid velocity when the hydrocarbon component of the steam cracking feed includes less-viscous, generally lighter, liquid-phase hydrocarbon. Generally, maintaining about 2% (weight basis) or greater of the hydrocarbon component of the steam cracking feed in the liquid phase, such as about 5% or greater, is sufficient to achieve sufficient flow velocity to maintain salt and/or particulate matter in suspension. At least a portion of the non-vapor-phase components of the steam cracking feed are in the liquid phase, and at least a portion of this liquid phase portion is conducted away with bottoms stream 119. For example, ≥10 wt. % of liquid-phase components of the steam cracking feed (including liquid-phase components that comprise one or more nitrogen-containing compositions), based on the weight of the steam cracking feed, can be conducted away with bottoms stream 119, such as ≥25 wt. %, or ≥50 wt. %, or ≥75 wt. %, or ≥90 wt. %. Likewise, at least a portion of the non-vapor-phase components of the steam cracking feed are in the solid or semi-solid phase (collectively, “solid phase”), and at least a portion of this solid phase portion is conducted away with bottoms stream 119. For example, ≥10 wt. % of solid-phase components of the steam cracking feed (including solid-phase components that comprise one or more nitrogen-containing compositions), based on the weight of the steam cracking feed, can be conducted away with bottoms stream 119, such as ≥25 wt. %, or ≥50 wt. %, or ≥75 wt. %, or ≥90 wt. %.

At least a portion of the steam cracking feed's nitrogen material is transferred to the separation vessel's bottoms stream, to be conducted away via line 119. Since the nitrogen material transferred to the bottoms stream is not present in the pyrolysis feed, the transferred nitrogen material will not be subjected to pyrolysis conditions in radiant coils 123 thus avoiding conversion of the transferred nitrogen material to one or more of ammonia, amine, acetonitriles, etc. In other words, separating from the steam cracking feed a pyrolysis feed having fewer (as compared to the steam cracking feed) nitrogen-containing compositions (particularly fewer of those in the liquid-phase and/or solid-phase) deceases the amounts of ammonia, amine, and/or acetonitriles in various process streams derived from the steam cracker effluent 125, e.g., decreasing the amount of acetonitrile in the C₄ and/or Pygas streams.

It is observed for a wide range of hydrocarbon feed comprising medium and/or heavy hydrocarbon (e.g., raw hydrocarbon such as crude oil) that selecting the specified process conditions for the desalter (when used), convection section, and vapor-liquid separator result in the conversion of ≤20 wt. % of the hydrocarbon feed's nitrogen-containing compositions (nitrogen atom basis) to the combined amount (nitrogen atom basis) of ammonia, amine, and acetonitrile in the steam cracker effluent. In some examples, one or more of the following is achieved: the amount of ammonia in the steam cracker effluent is in a range of about 10 wppm to about 100 wppm, the amount of acetonitrile in the steam cracker effluent is in a range of about 10 wppm to about 100 wppm, and the amount of amine in the steam cracker effluent is in a range of about 10 wppm to about 100 wppm. Certain other nitrogen-containing compositions that are transferred from the steam cracking feed to the pyrolysis feed are not converted to ammonia, amine, and/or acetonitrile in the steam cracker effluent. Typically these (or nitrogen-containing compositions derived therefrom) are removed from the process in the primary fractionator 141 and/or quench tower 147. When the pyrolysis feed includes appreciable amounts of both nitrogen-containing compositions and oxygen-containing compositions, the resulting NOx compounds in the steam cracker effluent are typically removed at locations downstream of the quench tower.

In certain aspects, the invention relates to optimizing at least two competing parameters: (i) the amount of desired products produced by the steam cracking, e.g., the amount of C₄₋ olefin, and (ii) the amount of nitrogen material in the pyrolysis feed to the convection coils, e.g., the amount of nitrogen-containing salts. It has surprisingly been found that for a wide range of hydrocarbon feeds comprising heavy hydrocarbon and nitrogen material, this optimization can be carried out by regulating the following separator process conditions: average temperature within the separator's separation zone, separator pressure, and flow rate of liquid in convection section coils located upstream of the separation vessel. Adjusting these process conditions to transfer a greater amount of the steam cracker feed to the pyrolysis feed favors the first parameter. Adjusting these process conditions to transfer a lesser amount of the steam cracker feed to the pyrolysis feed favors the second parameter

In these and other aspects, the separation vessel 117 typically operates at an average temperature (in the separation zone) in a range of about from about 315° C. to about 510° C. and/or a pressure from about 275 kPa to about 1,400 kPa, such as a temperature from about 430° C. to about 480° C., and/or a pressure from about 700 kPa to about 760 kPa. Typically, the flow velocity of liquid-phase material in convection coils located upstream of the vapor-liquid separator (namely those convention coils transporting steam cracking feed) is adjusted to maintaining about ≥2% (weight basis) of the hydrocarbon component of the steam cracking feed in the liquid phase, e.g., ≥3%, such as ≥5%, or ≥10%, or ≥15%, or more.

### Steam Cracker Radiant Section

The pyrolysis feed is transferred to the radiant section, where the pyrolysis feed is indirectly exposed (in one or more radiant coils) to the combustion carried out by the burners. As shown in FIG. 1, pyrolysis feed via line 121 is introduced into radiant line 123, where at least a portion of the pyrolysis feed's hydrocarbon is pyrolysed to produce steam cracker effluent, including C₂₊ olefins, which is transferred to line 125. The pyrolysis feed is typically in the vapor phase at the inlet of the radiant coils, e.g., about 90 wt. % or greater of the pyrolysis feed is in the vapor phase, such as about 95 wt. % or greater, or about 99 wt. % or greater.

Steam cracking conditions (pyrolysis conditions) may include exposing the pyrolysis feed in the radiant line 123 to a temperature (measured at the outlet of the radiant line) of about 400° C. or greater, such as, from about 400° C. to about 1,100° C., and a pressure of about 10 kPa or greater, and a steam cracking residence time from about 0.01 second to 5 seconds. For example, the steam cracking conditions can include one or more of (i) a temperature of about 760° C. or greater, such as from about 760° C. to about 1,100° C., or from about 790° C. to about 880° C., or for hydrocarbon feeds containing light hydrocarbon from about 760° C. to about 950° C.; (ii) a pressure of about 50 kPa or greater, such from about 60 kPa to about 500 kPa, or from about 90 kPa to about 240 kPa; and/or (iii) a residence time from about 0.1 seconds to about 2 seconds. The steam cracking conditions may be sufficient to convert at least a portion of the pyrolysis feed's hydrocarbon molecules to C₂₊ olefins by pyrolysis.

The steam cracker effluent generally includes unconverted pyrolysis feed and pyrolysis products. The pyrolysis products generally include the C₂₊ olefin, molecular hydrogen, acetylene, aromatic hydrocarbon, saturated hydrocarbon, C₃₊ diolefin, and one or more of aldehyde, acidic gases such as H₂S and/or CO₂, and mercaptan. The steam cracker effluent may be categorized as (i) vapor-phase products (i.e., products that would be primarily vapor-phase at 25° C. and a pressure of 1 bar absolute) such as one or more of acetylene, ethylene, propylene, butenes, and (ii) liquid-phase products (i.e., products that would be primarily liquid-phase at 25° C. and a pressure of 1 bar absolute) comprising, e.g., one or more C₅₊ hydrocarbon.

In certain aspects, the steam cracker effluent comprises molecular hydrogen, water (generally as steam), C₁-C₁₀ hydrocarbon, steam cracked gas oil (typically C₁₀-C₁₇ hydrocarbon), and SCT. In other aspects, the steam cracker effluent is a combination of molecular hydrogen, water (typically as steam), C₁-C₁₀ hydrocarbon, SCGO (typically a mixtures of C₁₀-C₁₂ hydrocarbon) having a normal boiling point range of about 174° C. to about 216° C., quench oil (typically C₁₂-C₁₇ hydrocarbon) having a normal boiling point range of about 216° C. to about 302° C., and SCT (typically C₁₇₊ hydrocarbon) having a normal boiling point range of about 302° C. to about 600° C. or more.

### Tar Knock Out Drum

Steam cracking process typically produce SCT, a relatively low-value, difficult to process composition, that can foul equipment under certain conditions. In general, hydrocarbon feeds containing a greater amount of higher boiling hydrocarbon tend to produce greater quantities of SCT. One way to decrease SCT formation includes rapidly decreasing steam cracker effluent temperature to a level at which the tar-forming reactions are greatly slowed. The rapid reduction in temperature of the steam cracker effluent may be achieved in one or more stages and using one or more methods and is referred to as quenching. The steam cracker effluent can be quenched by various methods such as contacting with cooled hydrocarbon (direct quench), or, alternatively, the steam cracker effluent can be rapidly cooled in heat exchangers.

As shown in FIG. 1, a tar knock-out drum 127, accepts steam cracker effluent (via line 125) and separates from the effluent SCT (which is transferred to line 129) and an upgraded steam cracker effluent (which is transferred to line 139). The steam cracker effluent may undergo cooling or quenching before being introduced to the tar knock-out drum or as it is introduced to the tar knock-out drum. Quenching can be carried out in one or more heat exchangers (not shown). Generally, the effluent leaving the first heat exchanger may remain at a temperature above the hydrocarbon dew point (the temperature at which the first drop of liquid condenses) of the steam cracker effluent. For a typical hydrocarbon feed containing heavy hydrocarbons under the indicated cracking conditions, the hydrocarbon dew point of the steam cracker effluent may be from about 375° C. to about 650° C., such as from about 480° C. to about 600° C. Above the hydrocarbon dew point, the fouling tendency is relatively low, because vapor phase fouling is generally not severe, and there is little to no liquid present that could cause fouling. The steam cracker effluent may be further cooled by one or more of (i) at least one additional heat exchanger, (ii) direct quench before reaching the tar knock-out drum, and (iii) direct quench within the tar knock-out drum.

In at least one embodiment, the steam cracker effluent is subjected to direct quench at one or more locations between the radiant line 123 and the tar knock-out drum 127. The quench is accomplished by contacting the steam cracker effluent with a liquid quench stream, in lieu of, or in addition to the treatment with transfer line exchangers. When employed in conjunction with at least one transfer line exchanger, the quench fluid may be introduced at a point downstream of the transfer line exchanger(s). Suitable quench fluids are typically sourced in the liquid phase, and at least partially vaporize upon contact with the steam cracker effluent. Conventional quench fluids can be used, but the invention is not limited thereto. Typical quench fluids include one or more quench oils, such as those separated from the steam cracker effluent or streams derived therefrom, e.g., quench oil separated in one or more of a tar knock-out drum, clean fuels unit, and primary fractionator. Alternatively or in addition, the quench fluid can include pyrolysis fuel oil and/or water, which can be obtained from various suitable sources, e.g., condensed dilution steam.

The temperature of the quenched steam cracker effluent entering the tar knock-out drum should be at a sufficiently low temperature to separate at least a portion of the SCT, e.g., a temperature of about 350° C. or less, such as in a range of from about 200° C. to about 350° C. or from about 240° C. to about 320° C.

Convention tar knock-out drums can be used, but the invention is not limited thereto. For example, tar knockout drum 127 can be a simple empty vessel, lacking distillation plates, trays, or stages. If desired, multiple knock-out drums may be connected in parallel such that individual drums can be taken out of service and cleaned while the plant is operating. The separated SCT typically has an initial boiling point ≥150° C., e.g., ≥200° C., such as in a range of from about 150° C. to about 320° C.

In one or more embodiments, a purge stream is introduced to the tar knock-out drum 127 to lessen liquid-vapor contact. When used, the purge stream can be, e.g., steam and/or substantially non-condensable hydrocarbons, such as those obtained from steam cracking, examples of which include cracked gas and tail gas. Surprisingly, it has been found that molecular nitrogen is an effective purge stream, and does not result in an appreciable increase in the amount of ammonia or NOₓ in the upgraded steam cracker effluent, even though the steam cracker effluent contains reactive hydrocarbon and oxygenate and the purging is carried out in a tar knock-out drum operating a temperature at which at least some NOₓ and ammonia formation reactions would be expected to occur.

In certain aspects, at least part of steam cracker effluent quenching is carried out in knock-out drum 127, e.g., by contacting (directly or indirectly, but typically directly) the steam cracker effluent through cool (less than 350° C.) quench fluid. cool quench fluid may be created by feeding a stream of SCT taken from the bottom of the tar knock-out drum 127 through a suitable heat exchanger (e.g., a shell-and-tube exchanger, spiral wound exchanger, airfin, or double-pipe exchanger) and recycling the cooled SCT stream to the tar knock-out drum 127. In at least one embodiment, sufficient cooled SCT is recycled to reduce the temperature of the SCT recycle from about 280° C. to about 150° C. The rate of asphaltene and tar formation in the line 125 and the tar knock-out drum 127 is greatly reduced at temperatures about 280° C. or less as compared to the higher temperatures of the steam cracker effluent when leaving the radiant coil 123. In another embodiment, the recycling suffices to lessen separated SCT viscosity to an extent sufficient to meet fuel oil viscosity specifications, in the absence or decrease of an added externally-sourced lower-viscosity blend stock that would otherwise necessary in the absence of said recycling. In another embodiment, the cooled SCT is introduced to tar knock out drum so as to provide an average temperature for SCT within the tar knock-out drum of about 175° C. or less, such as about 150° C. or less. Quenching methods may be adjusted to lessen or prevent the formation of asphaltenes. It may be possible to prevent formation of up to about 70 wt. % of asphaltenes through quenching the steam cracker effluent via line 125 in the tar knock-out drum 127.

### Clean Fuels Unit

The SCT from the tar knock-out drum can be further processed in a clean fuels unit, e.g., one or more a hydroprocessing units. For example, SCT, a utility fluid (optional), treat gas including molecular hydrogen, and catalyst can be combined under hydroprocessing conditions to produce clean fuels product (upgraded SCT) having improved blending characteristics with other heavy hydrocarbons such as fuel oil and blendstocks used to produce a fuel oil blend. The clean fuels unit may further remove at least a portion of nitrogen-containing impurities in the SCT, e.g., by hydroprocessing. For at least the reason the SCT and/or treat gas can comprise nitrogen material that can be converted under hydroprocessing conditions, ammonia is typically produced during the hydroprocessing. At least a portion of any ammonia, e.g., in hydroprocessor effluent, can be removed from the process by amine and/or caustic treating. In certain aspects shown in FIG. 1, a clean fuels unit 131 accepts a SCT 129 from a tar knock-out drum 127 and a lean amine stream via line 133. After hydroprocessing and removal of sulfur and other impurities, the clean fuels unit 131 produces a rich amine stream via line 135 and a clean fuels product stream via line 137.

SCT can be a highly aromatic product with a T₅₀ boiling point similar to a that of a vacuum gas oil and/or a vacuum resid fraction. SCT can be difficult to process using a fixed bed reactor because various molecules within the SCT are highly reactive, leading to fouling and operability issues. Such processing difficulties can be further complicated, for example, by the high viscosity of the SCT, the presence of coke fines, and/or other properties related to the composition of SCT.

The use of a utility fluid in hydroprocessing SCT has been observed lessen deposit formation and accumulation that would otherwise occur without utility fluid. The use of a utility fluid may provide a clean fuels product with a decreased viscosity, a decreased atmospheric T₅₀ and/or T₉₀ boiling point, and an increased hydrogen content over that of the SCT, resulting in improved compatibility with fuel oil and fuel oil blend-stocks. Additionally, hydroprocessing the SCT in the presence of utility fluid may produce fewer undesirable byproducts. Hydroprocessing the SCT in the presence of utility fluid has also been found to lessen the rate of increase in reactor pressure drop, which may increase run-length during hydroprocessing of SCT. Conventional utility fluids for SCT hydroprocessing can be used, but the invention is not limited thereto. For example, the utility fluid may be a portion of the clean fuels product that separated and recycled. Suitable processes for SCT hydroprocessing with a utility fluid and recycling a portion of the product stream as a utility fluid are disclosed in U.S. Pat. Nos. 9,777,227 and 9,809,756 and in International Patent Application Publication No. WO 2013/033590, which are incorporated herein by reference.

The relative amounts of utility fluid and SCT during hydroprocessing are generally from about 20 wt. % to about 95 wt. % of the SCT and from about 5 wt. % to about 80 wt. % of the utility fluid, based on total weight of utility fluid plus SCT. For example, the relative amounts of utility fluid and SCT during hydroprocessing can be, e.g., from about 20 wt. % to about 90 wt. % of the SCT and from about 10 wt. % to about 80 wt. % of the utility fluid, based on the combined weight of SCT+utility fluid conducted to the hydroprocessor, such as from about 40 wt. % to about 90 wt. % of the SCT and from about 10 wt. % to about 60 wt. % of the utility fluid. In an embodiment, the utility fluid:SCT weight ratio can be about 0.01 or greater, e.g., from about 0.05 to about 4, such as from about 0.1 to about 3, or from about 0.3 to about 1.1.

A utility fluid may include a solvent having significant aromatics content and generally, the utility fluid may also include a mixture of multi-ring compounds. The rings can be aromatic or non-aromatic and can contain a variety of substituents and/or heteroatoms. For example, the utility fluid can contain about 40 wt. % or greater, about 45 wt. % or greater, about 50 wt. % or greater, about 55 wt. % or greater, or about 60 wt. % or greater, based on the total weight of the utility fluid, of aromatic and non-aromatic ring compounds. The utility fluid can have an ASTM D86 10% distillation point of about 60° C. or greater and a 90% distillation point of about 350° C. or less. Optionally, the utility fluid (which can be a solvent or mixture of solvents) has an ASTM D86 10% distillation point of about 120° C. or greater, 140° C. or greater, or about 150° C. or greater and/or an ASTM D86 90% distillation point of about 300° C. or less.

The hydroprocessing is carried out in the presence of hydrogen by (i) combining molecular hydrogen with the SCT and/or utility fluid upstream of the hydroprocessing and/or (ii) conducting molecular hydrogen to the hydroprocessing as a separate hydroprocessor feed. Although relatively pure molecular hydrogen can be utilized for the hydroprocessing, it is generally desirable to utilize a “treat gas” which contains sufficient molecular hydrogen for the hydroprocessing and optionally other species (e.g., light hydrocarbon such as methane) which generally do not adversely interfere with or affect either the reactions or the products. The treat gas may contain about 50 vol % or greater of molecular hydrogen, such as about 75 vol % or greater, based on the total volume of treat gas conducted to the hydroprocessing stage.

The amount of molecular hydrogen supplied to the hydroprocessing stage can be from about 300 SCF/B (standard cubic feet per barrel) (53 S m³/m³) to about 5,000 SCF/B (890 S m³/m³), in which B refers to barrel of feed to the hydroprocessing stage (e.g., tar stream plus utility fluid). For example, the amount of molecular hydrogen can be from about 1,000 SCF/B (about 178 S m³/m³) to about 3,000 SCF/B (about 534 S m³/m³). Those skilled in the art will appreciate that the amount of molecular hydrogen supplied to the hydroprocessing may depend on the composition and properties of the SCT. For example, a lesser amount of molecular hydrogen can be supplied when the SCT contains a greater amount of C₆₊ olefin, for example, vinyl aromatics. Likewise, a greater amount of molecular hydrogen can be supplied when, e.g., the tar stream contains a relatively greater amount of sulfur-containing compositions.

At least part of the hydroprocessing of the clean fuels unit can be carried out in the presence of one or more hydroprocessing catalysts. Conventional hydroprocessing catalysts can be used, e.g., conventional homogeneous and/or heterogeneous catalysts, but the invention is not limited thereto. For example, suitable catalysts include those specified for use in SCT processing, resid processing, and/or heavy oil hydroprocessing, such as one or more of bulk (un-supported) catalysts, supported catalysts, and catalysts that form during hydroprocessing, e.g., those formed during hydroprocessing from precursors introduced upstream of the hydroprocessing. Examples of suitable hydroprocessing catalysts include one or more of KF860 available from Albemarle Catalysts Company LP, Houston Tex.; NEBULA® Catalyst, such as NEBULA® 20, available from the same source; CENTERA® catalyst, available from Criterion Catalysts and Technologies, Houston Tex., such as one or more of DC-2618, DN-2630, DC-2635, and DN-3636; ASCENT® Catalyst, available from the same source, such as one or more of DC-2532, DC-2534, and DN-3531; and FCC pre-treat catalyst, such as DN3651 and/or DN3551, available from the same source.

A wide range of hydroprocessing conditions can be used for SCT hydroprocessing, e.g., one or more of hydrocracking (including selective hydrocracking), hydrogenation, hydrotreating, hydrodesulfurization, hydrodenitrogenation, hydrodemetallation, hydrodearomatization, hydroisomerization, and hydrodewaxing. Hydroprocessing of the SCT in the presence of the utility fluid, treat gas, and catalyst can occur in one or more hydroprocessing stages, the stages comprising one or more hydroprocessing vessels or zones located downstream of the steam cracker and optionally downstream of the tar knock-out drum.

Catalytic hydroprocessing conditions can include, e.g., exposing the combined utility fluid and SCT to a temperature from about 50° C. to about 500° C., such as from about 200° C. to about 450° C., from about 220° C. to about 430° C., from about 300° C. to about 500° C., from about 350° C. to about 430° C., or from about 350° C. to about 420° C. proximate to the molecular hydrogen and hydroprocessing catalyst. Liquid hourly space velocity (LHSV) of the combined utility fluid and SCT may be from about 0.1 h⁻¹ to about 30 h⁻¹, or about 0.4 h⁻¹ to about 25 h⁻¹, or about 0.5 h⁻¹ to about 20 h⁻¹. For example, LHSV is about 5 h⁻¹ or greater, or about 10 h⁻¹ or greater, or about 15 h⁻¹ or greater. Molecular hydrogen partial pressure during the hydroprocessing can be from about 0.1 MPa to about 8 MPa, or about 1 MPa to about 7 MPa, or about 2 MPa to about 6 MPa, or about 3 MPa to about 5 MPa. In some embodiments, the partial pressure of molecular hydrogen is about 7 MPa or less, about 6 MPa or less, about 5 MPa or less, about 4 MPa or less, about 3 MPa or less, about 2.5 MPa or less, or about 2 MPa or less. The hydroprocessing conditions can include, a pressure from about 1.5 mPa to about 13.5 mPa, or from about 2 mPa to about 12 mPa, or from about 2 mPa to about 10 mPa. The hydroprocessing conditions may further include a molecular hydrogen consumption rate of about 53 standard cubic meters/cubic meter (S m³/m³) to about 445 S m³/m³ (300 SCF/B to 2500 SCF/B, where the denominator represents barrels of the tar stream, e.g., barrels of SCT).

When hydroprocessing SCT under the indicated conditions, the clean fuels product has improved properties compared to those of SCT and has greater utility than SCT as a fuel oil and/or fuel oil blending component. For example, the clean fuels product generally exhibits improved viscosity, solubility number, and insolubility number over the SCT and a lower sulfur content than SCT. Blending of the clean fuels product with other heavy hydrocarbons can be accomplished with little or no asphaltene precipitation, even without further processing of the clean fuels product prior to the blending.

If desired, one or more streams can be separated from the effluent of the hydroprocessor in the clean fuels unit, including from the clean fuels product, e.g., one or more overhead, mid-cut, and bottoms streams. Separation equipment can be configured for that purpose, e.g., one or more of distillation towers, vapor-liquid separators, splitters, fractionation towers, membranes, or absorbents. Describing the separated portions as overhead, mid-cut, and bottoms is not intended to preclude separation methods other than fractionating in a distillation tower. In certain aspects, one or more of the following streams are separated from the clean fuels product: an overhead stream that may include from about 0 wt. % to about 20 wt. % of the clean fuels product, a mid-cut stream that may include from about 20 wt. % to about 70 wt. % of the clean fuels product, and a bottoms stream that may include from about 20 wt. % to about 70 wt. % of the clean fuels product. One or more of these streams can be subjected to additional processing, e.g., to facilitate the removal of at least a portion of any remaining nitrogen-containing compositions and/or sulfur-containing compositions as might be present. Typically, at least the bottoms stream is subjected to hydroprocessing (e,g, in one or more hydroprocessing reactors located in the clean fuels unit or downstream of the clean fuels unit) to convert at least a portion of any nitrogen-containing compositions and/or sulfur-containing compositions in that stream. The hydroprocessing of the bottoms stream is typically carried out without added utility fluid, utilizing hydroprocessing conditions that are more severe than those utilized for producing clean fuels product. In certain aspects, e.g., those where a relatively light fuel oil and/or naphtha is desired, the bottoms stream hydroprocessing can include hydrocracking. The hydrocracking catalyst can be selected from among those having a nitrogen-tolerance, especially ammonia tolerance, e.g., when in aspects where the bottoms stream comprises an appreciable amount of nitrogen-containing impurities. Alternatively or in addition, other techniques may be used to protect the hydrocracking catalyst from deactivation by nitrogen-containing materials, e.g., one or more guard beds, sorbents, pre-reactors, etc. Conventional techniques can be used, but the invention is not limited thereto.

At least a portion of the overhead separated from effluent of one or more of the indicated hydroprocessing reactors can include used and unused treat gas, and may be recycled after removing at least a portion of any undesirable impurities such as H₂S and NH₃, e.g., by contacting with a lean amine solution and/or a lean caustic solution. The upgraded vapor product may be recycled as a portion of the treat gas. Furthermore molecular hydrogen may be added to recycled portion to maintain the level of hydrogen entering the clean fuels unit as necessary for SCT hydroprocessing. One advantage of the process is that in aspects which include the gas treating methods shown in FIG. 3B, treat gas for recycling to one or more of the hydroprocessing reactors can be upgraded in amine tower 305 and/or caustic tower 313, obviating the need for additional treat gas upgrading facilities.

### Primary Fractionator

Returning to FIG. 1, upgraded steam cracker effluent is conducted via line 139 to a separation stage, e.g., a primary fractionator 141 and a quench tower 147, for separation of a plurality of product, co-product, and by-product streams. Product streams may include one or more of (i) a primary fractionator bottoms stream (typically heavy hydrocarbon stream), which can be used, e.g., in one or more pumps-around of the primary fractionator and/or as quench oil, and can be transferred to line 143, (ii) SCGO, which is sent away via line 145, the SCGO including about 90 wt. % or greater of C₁₀-C₁₇ species based on the weight of the SCGO of material (e.g., C₁₀-C₁₇ hydrocarbon) having a T₉₀ in a range of from about 200° C. to about 290° C., (iii) Pygas, which is transferred via line 149 and contains C₅-C₁₀ hydrocarbons, and (iv) process gas, which is transferred via line 151.

One or more of these streams can have the following properties: (i) the heavy hydrocarbon stream of line 143 can be a quench oil comprising C₁₂-C₁₇ hydrocarbon, and can have a normal boiling point range of about 216° C. to about 302° C., (ii) the SCGO of line 145, can comprise about 90 wt. % or greater of C₁₀-C₁₂ hydrocarbon, based on the weight of the SCGO, and can have a T₉₀ in a range of about 174° C. to about 216° C., (iii) the Pygas of line 149 can comprise ≥90 wt. % of C₅₊ hydrocarbon, e.g., ≥90 wt. % of C₅-C₁₀ hydrocarbon, and (iv) a process gas, which is transferred via line 151. When a tar drum is not used upstream of the primary fractionator, the primary fractionator bottoms stream typically comprises SCT. In these aspects, the bottoms stream can comprise SCT in an amount ≥50 wt. %, based on the weight of the bottoms stream, e.g., ≥75 wt. %, such as ≥90 wt. %, or ≥95 wt. %. The SCT of the primary fractionator bottoms can have an initial boiling point ≥290° C., e.g., ≥350° C., such as ≥400° C., or ≥450° C., or ≥500° C., or ≥550° C., or even greater, and can comprise hydrocarbon compounds having an average molecular weight ≥212 g/mol.

Suitable primary fractionators and associated equipment are described in U.S. Pat. No. 8,083,931 and U.S. Pub. No. 2016/0376511, which are incorporated by reference herein. Additional stages for removing heat (such as one or more transfer line heat exchangers) and removing tar (such as tar drums) can be located in or upstream of the primary fractionator. Primary fractionator overhead can be conducted to quench tower 147 via line 142. The quench tower and primary fractionator can be combined in a single vessel, (e.g., with one located above the other), obviating the need for line 142, but this is not required.

The upgraded steam cracker effluent via line 139 is introduced to the primary fractionator 141 in a way that decreases contact with vapor-phase material in the fractionator, for more effective fractionation. If the upgraded steam cracker effluent were injected by spraying into the vapor space, the upgraded steam cracker effluent may warm, e.g., as a result of mixing with the large quantity of hot vapor present. This in turn may lead to an undesirable absorption into the sprayed upgraded steam cracker effluent of certain light hydrocarbon compounds residing in the vapor. Instead, the upgraded steam cracker effluent can be introduced near or preferably just below the liquid-vapor interface in the bottom of the primary fractionator. Introducing the upgraded steam cracker effluent below the vapor liquid interface ensures the stream is or stays cooled to the desired temperature and decreases the absorption of the indicated light components. An optional baffle placed above the vapor-liquid interface can lessen contact of the upgraded steam cracker effluent with hot vapor.

A primarily liquid-phase primary fractionator bottoms stream comprises heavy hydrocarbon, and can be removed from the primary fractionator via line 143. The primary fractionator bottoms can be combined with a primarily liquid-phase hydrocarbon blend stock of lesser viscosity and/or lesser temperature than the primary fractionator bottoms. Blendstock addition into a lower region of the primary fractionator can be used to control both the temperature (by cooling) and viscosity of the primary fractionator bottoms. Alternately, the blendstock may be added to the primary fractionator bottoms stream at a location downstream of the primary fractionator. The primary fractionator bottoms may be recycled to the primary fractionator at one or more locations as a pump-around and/or combined with the steam cracker effluent before the steam cracker effluent enters the tar knock-out drum (e.g., recycling to line 125). The primary fractionator bottoms can also be recycled to combine with the upgraded steam cracker effluent. In either manner, the primary fractionator bottoms can provide liquid cooling in the separations that occur in the tar knock-out drum or the primary fractionator.

The steam cracked gas oil may be condensed out of the vapor phase within the primary fractionator 141. Following disengagement and removal of liquid-phase material, remaining vapor constitutes a vapor phase effluent from the upper region of the primary fractionator. The vapor phase effluent (primary fractionator overhead) can be passed via line 142 and into one or more quench towers 147, where the vapor is rapidly cooled (quenched) as the vapor passes through water (vapor and/or liquid). The water can be obtained from a variety of sources, e.g., one or more of recycled refinery water, recirculated wastewater, clarified fresh water, purified wastewater, sour water stripper bottoms, overhead condensate, boiler feed water, and other water sources. Water is commonly recycled to the quench tower from downstream oil water separators, sour water separators, and Pygas strippers. The quench tower 147 condenses at least a portion of Pygas present in the primary fractionator overhead. Condensed Pygas and heated quench water are withdrawn from a location proximate to the bottom of the quench tower 147 as a Pygas stream.

Process gas (a primarily gaseous light hydrocarbon stream) is collected from the overhead of quench tower 147, and conducted away via line 151. When utilizing the specified pyrolysis feed and the specified steam cracker conditions, the process gas can include, for example, about 10 wt. % or greater of C₂₊ olefin, about 1 wt. % or greater of C₆₊ aromatic hydrocarbon, about 0.1 wt. % or greater of diolefin, saturated hydrocarbon, molecular hydrogen, acetylene, carbon dioxide, aldehyde, and C₁₊ mercaptan. The process gas may be directed to a light hydrocarbon recovery system for recovering light (e.g., C₂ to C₄) olefin, among other products, co-products, and by-products.

In some embodiments, the upgraded steam cracker effluent via line 139 is introduced into the primary fractionator 141 and the lower section of the quench tower 147 to produce at least the Pygas stream via line 149 and the process gas via line 151. In other embodiments, one or more fluids (e.g., a light effluent or a purified light effluent via line 237) can be flowed or otherwise transferred into line 142 and the quench tower 147, as further described with reference to FIG. 2.

### Oil Water Separator

In one or more embodiments, the pyrolysis process system 90 includes the Pygas and water separation and purification system 200, as depicted in FIG. 2. The Pygas stream via line 149 (also line 149 in FIG. 1) may be separated from water downstream in an oil and water separator 201 to form separated Pygas via line 203 and separated water containing nitrogen contaminants via line 217. A hydrocarbon-water mixture from via line 202 (also line 202 in FIG. 3A, and further described and discussed below) can also be introduced into the oil and water separator 201 and combined with the Pygas stream via line 149 and/or separated to form separated Pygas via line 203 and separated water via line 217.

The separated and concentrated Pygas (typically further comprising remaining water) may be transferred via line 203 for further processing in Pygas stripper 205. A purified Pygas is withdrawn from the bottoms portion of Pygas stripper 205 and may include C₅-C₁₀ hydrocarbon and is transferred via line 207 to a gasoline hydrogenation unit 209 to produce various naphtha boiling-range products (e.g., gasolines) via line 211. Water and light hydrocarbon can be removed from the top or overhead of the Pygas stripper 205, e.g., for recycling via line 213 to the primary fractionator. Water can be removed (not shown) from Pygas stripper 205, and may be transferred to downstream processes and/or conducted away.

Gasoline hydrogenation unit 209 typically includes one, two, three, or more stages for hydroprocessing the Purified pygas. The hydroprocessing can include, e.g., selective hydrogenation of Pygas diolefins to olefins. Nitrogen-containing compounds such as acetonitrile can spend, poison, or otherwise reduce the activity of catalysts contained in the various stages of the gasoline hydrogenation unit 209. Certain aspects of the invention avoid this difficulty by separating at least a portion of these contaminants from the Pygas of line 149, and conducting them away with the separated water via line 217. As such, the separated Pygas via line 203 and downstream products, such as the purified Pygas via line 207, can effectively be exposed to the catalysts in the gasoline hydrogenation unit 209 without spending, poisoning, and/or otherwise reducing catalyst activity.

At least a portion of any water separated from Pygas in oil and water separator 201 or from concentrated Pygas in stripper 205 may be removed, e.g., via lines 215 and/or 213, and recycled to the desalter, quench tower, or one or more steam generators. Although such steam can be used at various locations in pyrolysis process systems 90, this steam is typically conducted away to decrease the amount of nitrogen-containing compounds in in various streams of light hydrocarbon recovery system 300. The pH of separated water removed from oil and water separator 201 is typically regulated to be either neutral or acidic to increase the amount of nitrogen-containing compounds, e.g., ammonia, in solution. For example, the pH of water separated from oil and water separator 201 can be regulated to be (i) about 7 or less, such as about 4, about 4.5, or about 5 to about 5.5, about 6, about 6.5, about 6.8, or less than 7, or (ii) about 7.2, or about 7.4 to about 7.5, about 7.6, about 7.8, or about 8.

In certain aspects, at least a portion of the separated water stream is transferred through line 216 for purging or outgassing via a first purge fluid. The first purge fluid conducted away via line 216 typically includes nitrogen-containing compositions (e.g., ammonia, amine, etc.) and/or water. Alternatively or in addition, at least a portion of one or more of ammonia, amine, other nitrogen-containing compositions, hydrogen sulfide, and/or other non-aqueous impurities are removed from the separated water stream of line 217, e.g., by stripping in sour water stripper 219.

The pH of the separated water of line 217 is typically regulated to be ≥7, or ≥8, to help drive the ammonia-ammonium equilibrium toward favoring ammonia. This can be carried out by one or more of regulating the pH of the aqueous phase within separator 201 or at a location downstream thereof (e.g., by the addition of one or more compatible pH-controlling additives), regulating the amount of separated water conducted away via line 215, and regulating the amount of first purge fluid removed via line 216. The separated water of line 217 typically has (or is adjusted to achieve) a pH of about 7.2, about 7.5, about 7.8, about 8, or about 8.2 to about 8.5, about 8.8, about 9, about 9.2, about 9.5, about 9.8, about 10, or greater. For example, the separated water containing nitrogen contaminants via line 217 has a pH of greater than 7 to about 10, about 7.2 to about 10, about 7.5 to about 10, about 7.8 to about 10, about 8 to about 10, greater than 8 to about 10, about 8.2 to about 10, about 8.5 to about 10, about 8.8 to about 10, about 9 to about 10, greater than 7 to about 9, about 7.2 to about 9, about 7.5 to about 9, about 7.8 to about 9, about 8 to about 9, greater than 8 to about 9, about 8.2 to about 9, about 8.5 to about 9, or about 8.8 to about 9.

Light hydrocarbons and H₂S, for example, can be removed from the separated water of line 217 in one or more sour water strippers 219, and conducted away as components of a light effluent via line 221. Conventional sour water strippers can be used, but the invention is not limited thereto. Upgraded water removed as a bottoms stream from stripper 219 can be conducted away via line 223. At least a portion of the upgraded water can be conducted to one or more dilution steam generators 225 to provide steam via line 227 to steam cracking systems 100, e.g., as dilution steam for producing the stream cracking feed. The dilution steam generator may also produce nitrogen-laden aqueous stream as blow down, which can be removed via line 229. The blow-down typically comprises amine, and may further comprise other nitrogen-containing compositions. An advantage of various aspects of pyrolysis system 90 is that at least a portion of any amine present in that portion of the dilution steam of line 227 as is used to produce the steam cracking feed can be converted to more-easily-removed ammonia in steam cracking furnace 111 and removed at any of the one or more ammonia purge sites throughout the process system 90, e.g., in one or more purge streams of light hydrocarbon recovery system 300

In certain aspects it is desirable to remove nitrogen-containing compositions, such as ammonia, and acidic gases such as a hydrogen sulfide from the light effluent of line 221, e.g, by transferring one or more of these to a location for conduction away from the process. For example, valve 239 can be maintained in a closed or a partially closed position, which facilitates condensation of at least a portion of the light effluent in one or more condensers 231. The condenser is operated at a temperature of about 100° C. to about 150° C., about 110° C. to about 130° C., or about 115° C. to about 120° C., e.g., to condense at least a portion of any ammonia. The condensed effluent can be transferred to one or more containers, vessels, or drums 233, from which a second purge fluid can be conducted away via line 235. The second purge fluid typically comprises water and/or nitrogen-containing compositions, such as ammonia and/or amine. In these and other aspects, a purified light effluent conducted away via line 237 comprises a lesser amount of nitrogen-containing compositions such as ammonia and/or amine (based on the weight of the purified light effluent) than does the light effluent via line 221 (based on the weight of the light effluent). Typically, the purified light effluent of line 237 has an amount of nitrogen material that is about 5% less than that of the light effluent of line 221, such as about 8% less, or about 10% less to about 15% less, about 25% less, or about 50% less. In aspects where valve 239 is closed, line 235 carries away about 5%, about 10% or about 20% to about 30%, about 40%, or about 50% of the material in the separated water of line 217. When valve 239 is open or partially open, that portions of the light effluent in line 221 that is not condensed by condenser 231 can be transferred to quench tower 147 via lines 237 and 142.

The light effluent has a pH of greater than 7 or greater than 8 to help drive the ammonia-ammonium equilibrium to favouring ammonia. The light effluent has a pH of about 7.2, about 7.5, about 7.8, about 8, or about 8.2 to about 8.5, about 8.8, about 9, about 9.2, about 9.5, about 9.8, about 10, or greater. For example, the light effluent has a pH of greater than 7 to about 10, about 7.2 to about 10, about 7.5 to about 10, about 7.8 to about 10, about 8 to about 10, greater than 8 to about 10, about 8.2 to about 10, about 8.5 to about 10, about 8.8 to about 10, about 9 to about 10, greater than 7 to about 9, about 7.2 to about 9, about 7.5 to about 9, about 7.8 to about 9, about 8 to about 9, greater than 8 to about 9, about 8.2 to about 9, about 8.5 to about 9, or about 8.8 to about 9. In aspects that do not include condenser 231 and drum 233, the entire light effluent of line 221 can be conducted through the valve 239 (opened or partially opened), and from there via lines 237 and 142 to quench tower 147.

### Light Hydrocarbon Recovery System

FIGS. 3A and 3B schematically illustrate certain aspects of the invention that utilize a pyrolysis process system, such as system 90, which includes a light hydrocarbon recovery system 300. The invention is not limited to these aspects, and this description should not be interpreted as foreclosing other aspects of light hydrocarbon recovery within the broader scope of the invention. The process gas from the overhead of the quench tower is conducted via line 151 (from FIG. 1) for processing in one or more stages of a compressor-treatment system 310. The compressor-treatment system 310 includes one or more gas compressors 301, one or more condensers 302, and one or more knockout drums 303. Although FIG. 3A shows one each of gas compressor 301, condenser 302, and knockout drum 303 fluidly coupled in series within the compressor-treatment system 310, the invention is not limited thereto. In other aspects, the compressor-treatment system includes a plurality (e.g., two, three, four, or more) or more of fluidically-coupled compressor-condenser-knockout drum sets, typically coupled in series with each other (parallel coupling and series parallel coupling are within the scope of the invention).

The process gas from line 151 is compressed by gas compressor 301, condensed by condenser 302, and has portions removed by the knockout drum 303 which produces the compressed process gas of line 304. A hydrocarbon-water mixture is separated from the compressed process gas (and/or from partially-compressed process gas when knock out drum 303 is located between compression stages), and is conducted away via line 202. Knock-out drum(s) 303 can be a purged knock-out drum, i.e. one that is subjected to continuous, semi-continuous, periodic and/or intermittent purging, and in those aspects a third purge fluid is conducted away via line 306. The hydrocarbon-water mixture from line 202 can be transferred to the oil and water separator 201, as depicted in FIG. 2. The third purge fluid of line 306 is typically aqueous, and includes one or more nitrogen-containing compositions, such as ammonia and amine. As such, the compressed process gas of line 304 contains less nitrogen material (e.g., less ammonia and/or less amine) than does the process gas of line 151. For example, the compressed process gas of line 304 can have about 5% less nitrogen material (e.g., 5% less ammonia and/or 5% less amine) than does the process gas of line 151, e.g., about 10% less, or about 70% less, or about 90% less, such as in a range of about or about 20% less to about 50% less. Although the terms process gas, partially-compressed process gas, compressed process gas, partially-purified process gas, purified process gas, compressed purified process gas, upgraded process gas, etc. are used to describe streams derived from quench tower overhead at various stages of upgrading and purification, those skilled in the art will appreciate that referring to these streams as “gas” is a convenient label, but should not be interpreted as excluding liquid-phase material from one or more of these streams. Particularly after compression, at least a portion of one or more of these streams is typically liquid phase.

Although it is not required, the invention is compatible with combining the process gas (or one or more streams derived therefrom) with one or more streams from refinery and/or petrochemical process, e.g., processes for producing one or more of fuels, lubricating oils, and petrochemicals. Doing so has been found to be efficient, especially when the available refinery streams contain molecular hydrogen and/or C₂ to C₄ olefin. Excess capacity in process gas treatment and separation stages may occur, resulting, e.g., from initial over-design and/or during an interval of diminished process gas flow. This excess capacity can be utilized for (i) removing one or more desired products, e.g., C₂-C₄ olefin, from the indicated refinery and/or petrochemical streams and (ii) optionally recycling any remaining portion of the refinery and/or petrochemical streams (e.g., a portion comprising saturated hydrocarbon) for cracking as steam cracker furnace feed and/or combustion in steam cracker furnace burners, burners in other furnaces, etc. The process gas (or streams derived therefrom) can be combined with one or more refinery and/or petrochemical process streams upstream and/or downstream of compressor(s) 301, such as in one or more lines and/or vessels between compressor(s) 301 and fractionator 317. For example, one or more of the indicated refinery and/or petrochemical streams can be combined with one or more of process gas, partially-compressed process gas, and compressed process gas after, before, and/or between one or more stages of compressor(s) 301, e.g., in drum(s) 303.

As depicted in FIG. 3A, the compressed process gas is transferred via line 304 to an amine tower 305 for at least partial purification. Amine tower 305 accepts a lean amine stream 307. The lean amine stream is typically aqueous, and including one or more of ethanolamine, diethanolamine, methyldiethanolamine, diisopropanolamine, diglycolamine, and other amines Contacting the compressed process gas with the lean amine transfers acidic gases, e.g., hydrogen sulfide and carbon dioxide, from the process gas to the lean amine, which produces a rich amine stream that is conducted away via line 309. For further removal of acid gases, the partially-purified process gas after exiting the amine tower 305 may be passed through line 311 to a caustic tower 313, which may include aqueous hydroxide solutions, e.g., aqueous sodium hydroxide. The caustic tower 313 removes at least a portion of any remaining acid gases including hydrogen sulfide and carbon dioxide and also some weak acid gases (e.g., mercaptans) by transferring one or more of these from the partially-purified process gas to a lean caustic stream (not shown in FIG. 3A). This produces a purified process gas (conducted away via line 315) and a rich caustic stream (not shown in FIG. 3A).

Certain aspects of the amine and caustic treatments are shown schematically in FIG. 3B. The invention is not limited to these aspects, and this description should not be interpreted as excluding aspects in which the sequence of treatments is altered or even revered, or in which one of the treatments is omitted (e.g., when the hydrocarbon feed contains ≤1 wt. % of sulfur-containing compounds). As shown in FIG. 3B, the inlet streams of the amine tower 305 can include not only the compressed process gas of line 304 and the lean amine stream of line 307, but also an aqueous wash stream (“water wash”) via line 308. The outlet streams of the amine tower 305 include the partially-purified process gas of line 311, a fourth purge fluid via line 312, and a rich amine stream via line 314. The fourth purge fluid is typically aqueous, and can include one or more nitrogen-containing compositions such as ammonia and/or amine. As such, the partially-purified process gas of line 311 includes a lesser amount of nitrogen material, e.g., includes a lesser amount of ammonia, amine, and other nitrogen-containing compounds, than does the compressed process gas of line 304. The partially-purified process gas of line 311 can have, e.g., about 50% less nitrogen material (such as 50% less ammonia and/or 50% less amine) than does the compressed process gas of line 304, such as about 60% less, or about 90% less, or about 95% less, or in a range of about 70% less to about 80% less.

It is surprisingly and unexpectedly found that ammonia and/or other nitrogen contaminants are removed from the compressed process gas of line 304 into the amine tower 305 due to the relatively high pH value of the combined streams therein.

The rich amine stream is conducted via line 314 to one or more amine regenerators 316 for regeneration, which produces lean amine for recycle to the process Amine regenerator 316 has a lower section and an upper section, the lower section being that part of the regenerator that is at and below the second dashed line from the bottom, The upper section is that part of the regenerator that is at an above the third most dashed line from the bottom. Those skilled in the art will appreciate that dashed lines shown in various towers of FIGS. 1, 2, 3A, and 3B represent tower internals, e.g., trays, sheds, etc. used for facilitating the indicated separations. In certain aspects the regenerator includes a heating system 318 coupled to the lower section. The rich amine stream via line 314 is introduced into the amine regenerator 316 to produce the lean amine stream (removed via line 307) and a byproducts stream (removed via line 320), the byproducts stream containing acid and nitrogen material, including acidic nitrogen material. Lean amine stream of line 307 can be recycled to amine tower 305. The byproducts stream 320 can be cooled in one or more condensers 322 to produce a partially-condensed byproducts stream that is conducted away from the condenser via line 324. At least a portion of the vapor phase material (typically comprising acidic vapor) in the partially-condensed byproducts stream can be disengaged and removed via line 326 before introducing the remainder of the partially-condensed byproducts stream into one or more containers or drums 328. At least two streams can be removed from drum 328: a drum effluent comprising amine that is returned to regenerator 316 via line 330, e.g., as reflux, and a fifth purge fluid containing nitrogen material such as ammonia, the fifth purge fluid being conducted away via line 332.

The inlet streams of the caustic tower 313 include at least a portion of the partially-purified process gas via line 311 and the water wash via line 334. The outlet streams of the caustic tower 313 include at least a purified process gas via line 340 and a sixth purge fluid via line 336. The caustic tower 313 also includes a circulation system 338 containing one or more pumps and inlet and outlet conduits. The purge fluid via line 336 is typically aqueous, and can include nitrogen material such as ammonia and/or amine. As such, the purified process gas of line 340 includes a lesser amount of nitrogen material, e.g., includes a lesser amount concentrations of ammonia, amine, and other nitrogen-containing compounds, than does the partially purified process gas of line 311. The purified process gas of line 340 can have, e.g., about 5% less nitrogen material (such as 5% less ammonia and/or 5% less amine) than does the partially-purified process gas of line 311, such as about 10% less, or about 60% less, or about 80% less, or about 85% less, or about 90% less, or about 95% less, or in a range of about 20% less to about 40% less.

Acid may be included in the water wash of line 334, such by including a molar equivalent of acid per mole of basic compounds introduced into the caustic tower, e.g., with the partially-purified process gas. For example, a molar equivalent of acid may be added to the wash water upstream of caustic tower 313 per mole of basic nitrogen compounds (such as per mole of ammonia and/or per mole of amine) contained in the partially-purified process gas. Exemplary acids can be or include one or more of hydrochloric acid, sulfuric acid, phosphoric acid, monosodium hydrogen phosphate (MSHP), acetic acid, and salts of one or more of these. The amount of acid added to the partially-purified process gas can be regulated to achieve a pH of the purified process gas in line 340 that is neutral or basic.

Besides caustic and amine treatments, the light hydrocarbon recovery system 300 can include a compressor-treatment system 350. As depicted in FIG. 3B, the purified process gas can be conducted via line 340 to one or more stages of the compressor-treatment system 350. The compressor-treatment system 350 typically includes one or more gas compressors 342, one or more condensers 344, and one or more knockout drums 346. Although there can be one each of gas compressor 342, condenser 344, and knockout drum 346 fluidly coupled in series within the compressor-treatment system 350, other aspects (not shown) include a plurality (e.g., two, three, four, or more) of sets of the gas compressor 342, the condenser 344, and the knockout drum 346 sequentially and fluidly coupled with each other. Although the sets are typically coupled in series, this is not required, and in certain aspects the coupling is in parallel or series-parallel.

A compressed purified process gas produced by the compressor-treatment system 350 is introduced via line 348 into and passed through one, two, or more drier-ammonia beds 352. The drier-ammonia bed 352 can contains one, two, or more absorbent beds for removing ammonia and/or water to produce an upgraded process gas that is conducted away from the drier-ammonia bed via line 315. The drier-ammonia bed 352 can remove, e.g., about 0.5 wppm to about 50 wppm of ammonia from the compressed purified process gas, e.g., about 1 wppm to about 30 wppm, or about 2 wppm to about 20 wppm. As such, the upgraded process gas of line 315 has a lesser amount of ammonia and/or water than the compressed purified process gas of line 348. In one or more examples, the upgraded process gas can have, e.g., about 0.5% less ammonia that does the compressed purified process gas, e.g., about 1% less, about 4%, or about 5% less, or in a range of about 2% less to about 3%.

In some examples, the drier-ammonia bed 352 contains one, two, or more absorbent beds with activity for contaminant removal, such as activity for removing one or more of water, amine, and NOₓ. While contaminate removal is carried out in one, two, or more of the absorbent beds, other bed(s) can be taken off-line (withdrawn from contaminant-removal service) for at least partial regeneration. To do this, one or more lean regeneration gas streams are introduced via line 354 to a regenerating drier-ammonia bed 352. The lean regeneration gas regenerates one or more desiccant or molecular sieves beds (e.g., UOP-type N-Sieve) and/or the absorbent beds within the drier-ammonia beds 352 by removing from the regenerating beds at least a portion of one or more of the bed's contaminants, such as a portion of one or more of water, amine, and NOₓ. A rich regeneration gas (laden with contaminates, and typically containing at least some liquid and/or solid) is conducted away from the regenerating bed(s) for storage and/or further processing as a sixth purge gas via line 356. The sixth purge gas is typically aqueous and includes, e.g., one or more nitrogen materials such as ammonia and/or amine.

The upgraded process gas may be passed to one or more fractionation towers for separation and further purification of various hydrocarbon streams before further purification. Certain aspects which include separations of various streams from line 315 will now be described in more detail with continued reference to FIG. 3A. As shown, an initial separation is carried out in which first and second streams are separated from the upgraded process gas in a first fractionator 317: the first stream comprising molecular hydrogen, C₁-C₂ hydrocarbons, and some C₃₊ hydrocarbons and the second stream comprising C₃₊ hydrocarbon. The invention is not limited to these aspects, and this description should not be interpreted as excluding other aspects within the broader scope of the invention, such as aspects in which (i) the first stream comprises methane and molecular hydrogen, and the second stream comprises C₂₊ hydrocarbon, or (ii) the first stream comprises molecular hydrogen and C₃₋ hydrocarbon, and the second stream comprises C₄₊ hydrocarbon.

As shown in FIG. 3A, the first stream is removed from separation stage (e.g., a first fractionator) 317 via line 319 and the second stream is removed through line 321. A C₃ products stream (removed via line 325) and a C₄₊ products stream (removed via line 327) are separated from the second stream in second fractionator 323. Optionally, one or more water washes (typically liquid phase) of the C₄₊ products stream of line 327 can be used to lessen or eliminate nitrogen-containing compositions such as acetonitrile and/or other nitrogen-containing compounds. A C₄ products stream (removed via line 331) and a C₅₊ hydrocarbon stream (removed via line 333) are separated from the C₄₊ products stream of line 327 in a third fractionator 329. The C₅₊ hydrocarbon stream of line 207 (from the primary fractionator) and the C₅₊ hydrocarbon stream of line 333 are combined and may be passed through the gasoline hydrogenation unit 209 to produce various naphtha boiling-range products (e.g., one or more gasolines) that can be conducted away from the process via line 335. Since the C₄ products stream of line 331 can contain an appreciable amount of acetonitrile, e.g., particularly when the hydrocarbon feed to the steam cracker includes a heavy hydrocarbon such as crude oil, it can be advantageous to further process this stream to remove at least a portion of that nitrogen-containing compound. For example, the C₄ products stream can be condensed (e.g., by an indirect heat transfer against water), and then treated by contacting contacted with water. Doing so can remove at least a portion of any acetonitrile in the C₄ products stream of line 331, which can conducted away from the process, e.g., for storage and/or further processing. Those skilled in the art will appreciate that removing acetonitrile from the C₄ products stream may lessen the rate of catalyst deactivation during processes such as those which convert to MTBE and/or diisobutene at least a portion of isobutylene in the C₄ products stream.

The C₃ products stream of line 325 can be purified in columns that may include (i) a methanol/COS bed 337, then through line 339 to (ii) an arsine bed 341 to produce a reduced-arsine stream, which in turn passes through line 343 to (iii) an MAPD converter 345 for hydrogenation.

The reduced-arsine stream of line 343 and/or the purified C₃ stream of line 347 typically comprises little if any nitrogen-containing compositions. For example the these streams typically contain ammonia and/or other nitrogen-containing compounds in a total amount that is ≤1 wppm, such as ≤1 wppm, or in a range of about 0.001 wppm to about 0.8 wppm.

Propylene (transferred via line 351) and propane (transferred via line 353) can be separated from the purified C₃ hydrocarbons in fourth fractionator 349 (e.g., a C₃ splitter). The separated propane can be recycled for further cracking and/or conducted away, e.g., for storage and/or further processing. The propylene stream typically comprises few if any nitrogen-containing compositions. For example the this stream typically contains ammonia and/or other nitrogen-containing compounds in a total amount that is ≤1 wppm, such as about 0.001 wppm to about 0.8 wppm.

Returning again to the first stream removed from fractionator 317, this stream can be transferred through line 319 for further compression in compressor 355. The compressed first stream can be passed through line 357 to a series of purification stages, which may include one or more of (i) one or more beds 359 for removing sulfur-containing compositions (e.g., a mercaptan and carbonyl sulfide removal bed), then through line 361 to (ii) one or more beds 363 for removing arsine, and then through line 365 to (iii) one or more converters 367 for selectively converting C₂ acetylene to ethylene. A purified first stream is conducted via line 369 to demethanizer 371.

In demethanizer 371, an overhead stream comprising methane and a bottoms stream comprising C₂ hydrocarbon are separated from the purified first stream. The overhead stream is passed through line 373 to a cold box 375 in order to separate from the overhead stream (i) methane, which is conducted away via line 377 and (ii) molecular hydrogen, which is conducted away via line 379. The methane of line 377 may be used, e.g., as fuel gas and/or used as a feed and/or fuel for syngas generation. At least a portion of molecular hydrogen of line 379 can be recycled, e.g., to the clean fuels unit for use in one or more hydroprocessing units and/or (ii) for use in the acetylene and MAPD converters 367 and/or 345. The demethanizer bottoms stream may be passed through line 381 into a deethanizer 383 which removes residual C₃₊ and recycles the C₃₊ hydrocarbons through line 385 to line 325 and from there to methanol/COS bed 337. The overhead stream conducted away from fractionator 383 includes C₂ hydrocarbons and is passed through line 387 to a C₂ splitter 389 for separation from the overhead stream ethylene (transferred via line 391) and ethane (transferred via line 393). Ethane may be recycled for further cracking and/or conducted away such as for storage and/or further processing. The ethylene stream typically comprises few if any nitrogen-containing compositions. For example the this stream typically contains ammonia and/or other nitrogen-containing compounds in a total amount that is ≤1 wppm, such as in a range of about 0.001 wppm to about 0.8 wppm.

Each of the arsine beds 341, 363 independently contains one or more materials for removing arsine and/or other arsenic compounds, materials, or contaminants. For example, each of the arsine beds 341, 363 independently contains lead oxide which is used to remove arsine and/or other arsenic contaminants from the process stream upstream of converters containing catalyst beds, such as the MAPD converter 345 and/or the acetylene converter 367.

Overall, it has been found that removal of nitrogen-containing compositions from hydrocarbon feeds including heavy hydrocarbons (which can be useful for steam cracking) can be accomplished by one or more of: (i) purging, separating, and/or otherwise removing one or more nitrogen-containing compositions from a separated water component in a line downstream of an oil and gas separator and upstream of a sour water stripper, (ii) purging, separating, and/or otherwise removing one or more nitrogen-containing compositions from an overhead stripping of a light effluent in a line downstream of a sour water stripper and downstream of one of more condensers, (iii) purging, separating, and/or otherwise removing one or more nitrogen-containing compositions from a process gas (e.g., quench tower gaseous overhead) that is collected from an overhead of a quench tower, and then passing the process gas through one or more compressors, one or more condensers, and one or more knockout drums or vessels, (iv) purging, separating, and/or otherwise removing one or more nitrogen-containing compositions from a compressed or partially-compressed process gas within one or more amine towers, one or more amine regenerators, and/or one or more caustic towers, and/or (v) purging, separating, and/or otherwise removing one or more nitrogen-containing compositions from a purified process gas by one or more drier-ammonia beds to produce a upgraded process gas.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure, additionally, they do not exclude impurities and variances normally associated with the elements and materials used.

For brevity and clarity, the following generalities apply. Each cited document is incorporated by reference herein, including any testing procedures to the extent they are not inconsistent with this text. Although certain forms and aspects have been illustrated and described, various modifications can be made without departing from the spirit and scope of the invention. The term “comprising” is considered synonymous with the term “including”. Whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa. The ranges of this description encompass any lower limit combined with any upper limit. Likewise, (i) ranges from any lower limit may be combined with any other lower limit, and (ii) ranges from any upper limit may be combined with any other upper limit. The ranges of this description include every point or individual value between its end points. The ranges include every point or individual value may serving as its own lower or upper limit combined with any other point or individual value or any other lower or upper limit.

1. A steam cracking method, comprising
providing a hydrocarbon feed comprising hydrocarbon and a first nitrogen material;
introducing the hydrocarbon feed to a steam cracker to produce a steam cracker effluent;
separating from the steam cracker effluent a steam cracker tar and an upgraded steam cracker effluent;
separating from the upgraded steam cracker effluent (i) a process gas comprising a second nitrogen material and (ii) a Pygas comprising a third nitrogen material, wherein the second and third nitrogen materials are each a portion of the first nitrogen material and/or are each derived from a portion of the first nitrogen material;
separating from the Pygas stream a concentrated Pygas and a separated water component containing at least a portion of the third nitrogen material;
separating from the separated water component a light effluent and a remaining water component, wherein the light effluent comprises at least a portion of the third nitrogen material; and
removing at least a portion of the third nitrogen material in the light effluent to produce a purified light effluent.

2. The method of claim 1, wherein the separation of at least a portion of the light effluent's nitrogen material includes:
condensing at least a portion of the light effluent's nitrogen material and/or condensing at least a portion of the purified light effluent's nitrogen material; and
transferring at least a portion of the condensed nitrogen material to at least one vessel.

3. The method of claim 2, wherein the condensation occurs at a temperature in a range of about 100° C. to about 150° C.

4. The method according claim 3, wherein the condensation occurs at a temperature in a range of about 120° C. to about 130° C.

5. The method according to claim 1, wherein the separation of the Pygas stream and the process gas is carried out in at least one primary fractionator and/or in at least one quench tower, and further comprising transferring at least a portion of the purified light effluent to the quench tower.

6. The method according to claim 1, wherein (i) the separation of the concentrated Pygas and the separated water component is carried out in an oil and water separator, (ii) the separation of the light effluent and the remaining water component is carried out in a water stripper, (iii) the separated water component comprises at least a portion of the third nitrogen material; and further comprising removing from the separated water component at least a portion of the separated water component's portion of the third nitrogen material at a location downstream of the oil and water separator and upstream of the water stripper.

7. The method according to claim 1, further comprising:
transferring the process gas through a compressor and a condenser and into a knockout drum to produce a compressed process gas comprising first portion of the second nitrogen material, a hydrocarbon-water mixture, and a purge fluid comprising a second portion of the second nitrogen material.

8. The method of claim 7, further transferring to an amine solution in an amine tower at least a first portion of the compressed process gas's nitrogen material to produce a partially-purified process gas.

9. The method of claim 8, further comprising circulating an amine solution between the amine tower and an amine regenerator.

10. The method of claim 9, further comprising removing from the amine solution at least a part of the second nitrogen material in the amine solution in the amine regenerator.

11. The method of claim 9, further comprising transferring away from the partially-purified process gas at least part of the second nitrogen material in the partially-purified process gas to produce a purified process.

12. The method of claim 11, wherein the transfer of at least a portion of the partially-purified process gas's second nitrogen material is carried out using a water wash within a caustic tower.

13. The method of claim 12, further comprising combining acid with the water wash.

14. The method of claim 11, further compressing the purified process gas.

15. The method of claim 14, further comprising flowing the compressed purified process gas through a drier-ammonia bed to transfer to the drier-ammonia bed at least a portion of any remaining second nitrogen material in the purified process gas to produce an upgraded process gas.

16. The method of claim 15, further comprising removing from the drier-ammonia bed at least a portion of the transferred second nitrogen material.

17. The method of claim 15, further comprising (i) separating olefin from purified process gas and (ii) polymerizing at least a portion of the separated olefin.

18. The method according to claim 1, wherein the portion of the third nitrogen material in the light effluent comprises one or more of ammonia, ammonium. amine, nitrile, hydrogen cyanide, one or more NOₓ, compounds, and one or more ions and/or salts of NOₓ, compounds.

19. A method for producing light olefins from a feed comprising heavy hydrocarbon and a first nitrogen material, comprising:
introducing a hydrocarbon feed to a steam cracker to produce a steam cracker effluent;
separating from the steam cracker effluent a steam cracker tar and an upgraded steam cracker effluent;
separating from the upgraded steam cracker effluent at least (i) a process gas comprising a second nitrogen material and (ii) a Pygas comprising a third nitrogen material, wherein the second and third nitrogen materials are each a portion of the first nitrogen material and/or are each derived from a portion of the first nitrogen material;
transferring the process gas through a compressor and a condenser and into a knockout drum to produce a compressed process gas comprising first portion of the process gas's second nitrogen material, a hydrocarbon-water mixture, and a purge fluid comprising a second portion of the process gas's second nitrogen material; and
flowing the compressed process gas through an amine tower and a caustic tower to produce a purified process gas.

20. The method of claim 19, further comprising removing at least a portion of the compressed process gas's second nitrogen material in the amine tower, the caustic tower, or a combination thereof.

21. The method of claim 20, further comprising:
circulating an amine solution between the amine tower and an amine regenerator, wherein the amine solution comprises at least a portion of the second nitrogen material removed from the compressed process gas; and
removing at least a portion of the amine solution's second nitrogen material from the amine regenerator.

22. The method according to claim 19, further comprising:
compressing the purified process gas;
removing transferring at least a portion of any second nitrogen material in the compressed purified process gas to a at least one drier-ammonia bed to produce an upgraded process gas; and
conducting away from the drier-ammonia bed at least a portion of the transferred second nitrogen material.

23. The method according to claim 19, wherein the second nitrogen material includes ammonia and/or ammonium.

24. The method according to claim 19, wherein the second nitrogen material includes one or more of amine; nitrile; hydrogen cyanide; one or more NOₓ compounds; one or more ions of NOₓ compounds, and one or more salts of NOₓ compounds.

25. A heavy-hydrocarbon conversion process, comprising:
introducing a feed to a steam cracker to produce a steam cracker effluent, wherein the feed comprises heavy hydrocarbon and a first nitrogen material;
separating from the steam cracker effluent in at least one tar knock-out drum at least a steam cracker tar and an upgraded steam cracker effluent;
separating from the upgraded steam cracker effluent at least a (i) a process gas containing a second nitrogen material and (ii) a Pygas containing a third nitrogen material, wherein the separation is carried out in a primary fractionator and/or quench tower, the second nitrogen material is a portion of the first nitrogen material and/or is derived from a portion of the first nitrogen material, and the third nitrogen material is a portion of the first nitrogen material and/or is derived from a portion of the first nitrogen material;
compressing the process gas and separating from the compressed and/or partially-compressed process gas a purge fluid comprising a portion of the second nitrogen material;
contacting the compressed process gas with a lean amine composition in at least one amine tower to produce a rich amine composition and a partially-purified process gas;
contacting the partially-purified process gas with a lean caustic composition in at least one caustic tower to produce a rich caustic composition and a purified process gas;
removing at least a portion of any of the second nitrogen material in the rich amine composition in at least one amine regenerator to produce a regenerated amine composition, and recycling at least a portion of the regenerated amine composition as the lean amine composition;
removing in a at least one drier-ammonia bed at least a portion of any remaining second nitrogen material in the purified process gas to produce an upgraded process gas;
conducting away from the drier-ammonia bed at least a portion of the second nitrogen material removed from the purified process gas;
separating a water component from the Pygas stream in at least one oil and water separator to produce a concentrated Pygas;
separating a light effluent and a remaining water component from the separated water component in a least one stripper, to produce a light effluent and a remaining water component, wherein the light effluent comprises at least a portion of the third nitrogen material; and
separating from the light effluent at least a portion of the third nitrogen material to produce a purified light effluent.

26. The process of claim 25, further comprising (i) separating at least a C₄ stream from the low-ammonia hydrocarbon stream, and (ii) contacting at least part of the C₄ stream with water to produce an upgraded C₄ stream comprising isobutene, wherein at least a portion of any acetonitrile in the C₄ stream is transferred to the water.

27. The process of claim 26, further comprising catalytically converting at least a portion of the isobutene to diisobutene and/or MTBE.

28. A system for managing nitrogen material during steam cracking of a crude feed comprising heavy hydrocarbons, the system comprising:
a steam cracker comprising a convection line and a radiant line there within;
a flash separation vessel fluidly coupled to and downstream of the convection line and fluidly coupled to and downstream of the radiant line;
a tar knock-out drum fluidly coupled to and downstream of the radiant line;
a fractionator fluidly coupled to and downstream of the tar knock-out drum;
a quench tower fluidly coupled to and downstream of the fractionator;
an oil and water separator fluidly coupled to and downstream of the quench tower; and
a water stripper fluidly coupled to and downstream of the oil and water separator, wherein the water stripper comprises an overhead which is fluidly coupled to and upstream of a condenser and a vessel by a first line and fluidly coupled to and upstream of the quench tower by a second line.
